(19) United States
(12) Patent Application Publication
(10) Pub. No.: US 2011/0003890 A1
(43) Pub. Date: Jan. 6, 2011

(54) USE OF ANGIOGENESIS ANTAGONISTS IN CONDITIONS OF ABNORMAL VENOUS PROLIFERATION

(76) Inventors: Jason Joel Schwartz, Salt lake City, UT (US); Thomas P. Kennedy, Charlotte, NC (US)

Correspondence Address: Ballard Spahr LLP, SUITE 1000, 999 PEACHTREE STREET, ATLANTA, GA, 30309-3915, US

(21) Appl. No.: 12/741,979

(22) PCT Filed: Nov. 10, 2008
(86) PCT No.: PCT/US08/83028

§ 371 Date: Sep. 14, 2010

Related U.S. Application Data
(60) Provisional application No. 60/986,362, filed on Nov. 8, 2007.

(51) Int. Cl. A61K 31/27; A61K 31/315; A61P 1/16

(52) U.S. Cl. 514/491; 514/476

(57) ABSTRACT

The present application describes therapy with angiogenesis antagonists such as anti-VEGF antibodies. In particular, the application describes the use of such angiogenesis antagonists to treat end-stage liver disease and end-stage liver disease complications. The present application also describes the use of such angiogenesis antagonists to treat disorders of altered venous proliferation such hemorrhoids and varicose veins.

### CROSS-REFERENCE TO RELATED APPLICATIONS

This application claims benefit of U.S. Provisional Application No. 60/986,362, filed Nov. 8, 2007, which is hereby incorporated herein by reference in its entirety.

### BACKGROUND

According to the World Health Organization, 10% of the world's population has chronic liver disease, including 25 million Americans. In China alone, half a million die of the disease each year. The majority of chronic liver disease can be attributed to viral hepatidides, primarily hepatitis B, which infects 2 billion people worldwide, 350 million of which have developed life-long infections. Similarly, it is estimated that approximately 300 million individuals are infected with the hepatitis C virus, with 3-4 million new cases occurring each year. Eighty percent of these individuals can be expected to develop chronic infections, and 10-20% can be expected to develop cirrhosis. In the United States, cirrhosis is the 4th leading cause of death in patients aged 45-64, accounting for 1.5 billion dollars in direct health care costs in 2000 and $1.1 billion in facility costs. At the present time, in patients admitted to the hospital for complications of cirrhosis, 10% can be expected to die while hospitalized.

Not much is known regarding the pathophysiology of cirrhosis, a disease characterized by replacement of functioning liver parenchyma with fibrotic scar tissue and eventual loss of liver function. As a consequence of altered flow, pressure, and resistance in the portal circulation, patients with cirrhosis experience an inexorable decline in multiple organ systems leading to loss of vitality, productivity, and death. One of the leading suppositions regarding the pathogenesis of end-stage liver disease invokes the “peripheral vasodilatation theory.” This theory seeks to explain the cause of the intense underfilling of the central circulation and hyperdynamic physiology observed in patients with cirrhosis. According to this theory, a portion of the effective circulating blood volume is sequestered in the venous capacitance vessels of the splanchnic vasculature for reasons not completely understood.

For decades, it has been understood that these venous capacitance vessels are not only increased in size, but also in number in patients with end-stage liver disease. It is this propensity to form venous collaterals (varices) which impedes the surgeon's ability to operate safely in this patient population as a consequence of the massive hemorrhage which may ensue. These abnormal vessels form in all organs in direct continuity with the portal circulation and not only bleed profusely at the time of surgery, but may also rupture externally, causing exsanguinating gastrointestinal hemorrhage, a major cause of death in the cirrhotic population. It is reasonable to assume that the presence of these vessels may also contribute directly to the underfilling seen in accordance with the “peripheral vasodilatation theory,” resulting in many of the manifestations of cirrhotic liver disease including abnormalities of sodium handling, renal failure, ascites, encephalopathy, and hepatopulmonary syndrome, amongst others.

Similar to other models of human disease involving abnormal blood vessel formation such as macular degeneration, one may posit an angiogenic basis for the portal-systemic collaterals which form in the setting of portal hypertension. Indeed, monoclonal antibodies against vascular endothelial growth factor receptor (VEGF)-2 have been shown to prevent portal-systemic collateral formation in portal—hypertensive mice (Fernandez et al. Hepatology. Jul. 24 (2007) and increases in portal pressure have been shown to upregulate VEGF in the intestinal microcirculatory bed (Abraldes et al.). Similarly, endothelial cells exposed to varying degrees of increased pressure have also been found to express increased levels of VEGF (Suzuma et al.) and rats with cirrhosis and portal hypertension have been found to develop increased angiogenesis in conjunction with increased levels of VEGF expression (Geerts et al.). Finally, patients with portal hypertensive gastropathy, a complication of end-stage liver disease, have also been found to have increased levels of VEGF expression (Tsugawa et al.).

As there are no known effective therapies for end-stage liver disease aside from liver transplantation and donor organs remain in severe shortage, the practical implications of a therapy shown to be even marginally successful are of great interest. From a commercial standpoint, use of anti-angiogenic therapy in the end-stage liver disease population has the potential for use in 5-10% of the world's population. Two other diseases of abnormal venous proliferation include varicose veins and hemorrhoids, two of the most ubiquitous disorders of the human condition. Because the potential therapeutic market for anti-angiogenic therapies in the setting of portal hypertension and diseases of venous proliferation dwarfs the current oncologic applications of anti-angiogenesis drugs like bevacizumab, the clinical implications of their use in these populations is staggering.

Angiogenesis is an important cellular event in which vascular endothelial cells proliferate, prune and reorganize to form new vessels from preexisting vascular network. There are compelling evidences that the development of a vascular supply is essential for normal and pathological proliferative processes (Folkman and Klagsbrun (1987) Science 235:442-447). Delivery of oxygen and nutrients, as well as the removal of catabolic products, represent rate-limiting steps in the majority of growth processes occurring in multicellular organisms. Thus, it has been generally assumed that the vascular compartment is necessary, albeit but not sufficient, not only for organ development and differentiation during embryogenesis, but also for wound healing and reproductive functions in the adult.

Angiogenesis is also implicated in the pathogenesis of a variety of disorders, including but not limited to, proliferative retinopathies, age-related macular degeneration, tumors, autoimmune diseases such as rheumatoid arthritis (RA), and psoriasis. Angiogenesis is a cascade of processes consisting of 1) degradation of the extracellular matrix of a local venue after the release of protease, 2) proliferation of capillary endothelial cells, and 3) migration of capillary tubules toward the angiogenic stimulus. Ferrara et al. (1992) Endocrine Rev. 13:18-32.

In view of the remarkable physiological and pathological importance of angiogenesis, much work has been dedicated to the elucidation of the factors capable of regulating this process. It is suggested that the angiogenesis process is regulated by a balance between pro- and anti-angiogenic molecules, with various disease states, especially cancer, having the capacity to exert considerable influence on tightly-regulated pathways. (Carmeliet and Jain (2000) Nature 407:249-257).

Vascular endothelial cell growth factor (VEGF), a potent mitogen for vascular endothelial cells, has been reported as a pivotal regulator of both normal and abnormal angiogenesis. Ferrara and Davis-Smyth (1997) Endocrine Rev. 18:4-25; Ferrara (1999) J. Mol. Med. 77:527-543. Compared to other growth factors that contribute to the processes of vascular formation, VEGF is unique in its high specificity for endothelial cells within the vascular system. Recent evidence indicates that VEGF is essential for embryonic vasculogenesis and angiogenesis. Carmeliet et al. (1996) Nature 380:435-439; Ferrara et al. (1996) Nature 380:439-442. Furthermore, VEGF is required for the cyclical blood vessel proliferation in the female reproductive tract and for bone growth and cartilage formation. Ferrara et al. (1998) Nature Med. 4:336-340; Gerber et al. (1999) Nature Med. 5:623-628.

In addition to being an angiogenic factor in angiogenesis and vasculogenesis, VEGF, as a pleiotropic growth factor, exhibits multiple biological effects in other physiological processes, such as endothelial cell survival, vessel permeability and vasodilation, monocyte chemotaxis and calcium influx. Ferrara and Davis-Smyth (1997), supra. Moreover, recent studies have reported mitogenic effects of VEGF on a few non-endothelial cell types, such as retinal pigment epithelial cells, pancreatic duct cells and Schwann cells. Guerrin et al. (1995) J. Cell Physiol. 164:385-394; Oberg-Welsh et al. (1997) Mol. Cell. Endocrinol. 126:125-1312; Sondell et al. (1999) J. Neurosci. 19:5731-5740.

Substantial evidence also implicates VEGF's critical role in the development of conditions or diseases that involve pathological angiogenesis. The VEGF mRNA is overexpressed by the majority of human tumors examined (Berkman et al. J Clin Invest 91:153-159 (1993); Brown et al. Human Pathol. 26:86-91 (1995); Brown et al. Cancer Res. 53:4727-4735 (1993); Mattem et al. Brit. J. Cancer. 73:931-934 (1996); and Dvorak et al. Am J. Pathol. 146:1029-1039 (1995)). Also, the concentration of VEGF in eye fluids is highly correlated to the presence of active proliferation of blood vessels in patients with diabetic and other ischemia-related retinopathies (Aiello et al. N. Engl. J. Med. 331:1480-1487 (1994)). Furthermore, recent studies have demonstrated the localization of VEGF in choroidal neovascular membranes in patients affected by AMD (Lopez et al. Invest. Ophtalmo. Vis. Sci. 37:855-868 (1996)).

The recognition of VEGF as a primary regulator of angiogenesis in pathological conditions has led to numerous attempts to block VEGF activities Inhibitory anti-VEGF receptor antibodies, soluble receptor constructs, antisense strategies, RNA aptamers against VEGF and low molecular weight VEGF receptor tyrosine kinase (RTK) inhibitors have all been proposed for use in interfering with VEGF signaling (Siemeister et al. Cancer Metastasis Rev. 17:241-248 (1998). Indeed, anti-VEGF neutralizing antibodies have been shown to suppress the growth of a variety of human tumor cell lines in nude mice (Kim et al. Nature 362:841-844 (1993); Warren et al. J. Clin. Invest. 95:1789-1797 (1995); Borgstrom et al. Cancer Res. 56:4032-4039 (1996); and Melnyk et al. Cancer Res. 56:921-924 (1996)) and also inhibit intraocular angiogenesis in models of ischemic retinal disorders (Adamis et al. Arch. Opthalmol. 114:66-71 (1996)). Therefore, anti-VEGF monoclonal antibodies or other inhibitors of VEGF action are promising candidates for the treatment of solid tumors and various intraocular neovascular disorders. Although the VEGF molecule is upregulated in tumor cells, and its receptors are upregulated in tumor infiltrated vascular endothelial cells, the expression of VEGF and its receptors remain low in normal cells that are not associated with angiogenesis. Thus, such normal cells would not be affected by blocking the interaction between VEGF and its receptors to inhibit tumor angiogenesis, and therefore tumor growth and cancer metastasis.

Monoclonal antibodies are now commonly manufactured using recombinant DNA technology. Widespread use has been made of monoclonal antibodies, particularly those derived from rodents. However, nonhuman antibodies are frequently antigenic in humans. The art has attempted to overcome this problem by constructing “chimeric” antibodies in which a nonhuman antigen-binding domain is coupled to a human constant domain (Cabilly et al., U.S. Pat. No. 4,816,567). The isotype of the human constant domain may be selected to tailor the chimeric antibody for participation in antibody-dependent cellular cytotoxicity (ADCC) and complement-dependent cytotoxicity. In a further effort to resolve the antigen binding functions of antibodies and to minimize the use of heterologous sequences in human antibodies, humanized antibodies have been generated for various antigens in which substantially less than an intact human variable domain has been substituted by the corresponding sequence from a non-human species have substituted rodent (CDR) residues for the corresponding segments of a human antibody to generate. In practice, humanized antibodies are typically human antibodies in which some complementarity determining region (CDR) residues and possibly some framework region (FR) residues are substituted by residues from analogous sites in rodent antibodies. Jones et al., Nature 321:522-525 (1986); Riechmann et al., Nature 332:323-327 (1988); Verhoeyen et al., Science 239:1534-1536 (1988).

Several humanized anti-human VEGF (hVEGF) antibodies have been successfully generated, and have shown significant hVEGF-inhibitory activities both in vitro and in vivo. Presta et al. (1997) Cancer Research 57:4593-4599; Chen et al. (1999) J. Mol. Biol. 293:865-881. One specific humanized anti-VEGF antibody, bevacizumab (Avastin.RTM., Genentech, Inc.), has been approved in the US for use in combination with chemotherapeutic agents for treating metastatic colorectal cancer (CRC). The drug is currently used in several clinical trials for treating various other cancers. Another high-affinity variant of the humanized anti-VEGF antibody is currently clinically tested for treating age-related macular degeneration (AMD).

Despite recent developments in anti-angiogenic therapies in the fields of oncology, arterial remodeling, and disorders of arterial proliferation, disorders of abnormal venous proliferation have not historically been included within disease models alluding to an angiogenic paradigm. Among the reasons for lack of attention to this potential area of use has been the poor understanding of venous diseases, in particular, chronic liver disease, a failure to appreciate their impact in diseases common to the human condition, and an assumption that “veins are arteries”. The assumption of homology between the cellular physiology of arteries and veins is, however, erroneous. Not only is there a well-known ultrastructural difference between arteries and veins, with arteries known to possess a well-developed media, but there are also well-described phenotypic differences at the cellular level as well. For instance, significant disparity exists with regard to gene expression when endothelial cells from different vascular beds are exposed to homeostatic imbalance. Additionally, these cells have been shown to demonstrate distinct responses to altered conditions of pressure, shear stress, and inflammatory stimuli (Wagner, Henderson et al. 1988; Upchurch, Banes et al. 1989; Iba, Maitz et al. 1991; Quist, Haudenschild et al. 1992; Quist and LoGerfo 1992; Garcia-Cardena, Comander et al. 2001; Faries, Rohan et al. 2002; Wong, Nili et al. 2005; Cooley, Chen et al. 2007; Seebach, Donnert et al. 2007).

Up to now, it has been difficult to invoke an angiogenic basis for disorders of abnormal venous proliferation. However, integration of accumulated knowledge from a wide array of disparate and unrelated sources has allowed for the advancement of a novel theory of venogenesis not previously defined in the art. At its core, is the understanding that alterations in flow, related to Ohm's Law, ΔP=Q×R, where P is pressure, Q is flow, and R is resistance, are directly responsible for inducing venogenesis. This theory contends that alterations in this relationship influence the formation of abnormal venous collaterals without regard to etiology. In the end-stage liver disease patient, the inciting factor is superfluous as these altered relationships represent a final common pathway, one in which an increase in venous capacitance and a reduction in circulating blood volume leads to the hyperdynamic circulation, abnormal sodium handling, variceal hemorrhage, ascites, encephalopathy, coma, and renal failure, before death finally ensues.

In models far removed from the clinical arena, alterations in vascular blood flow and pressure have been found to act on endothelial cells in vitro (Fung and Liu 1993; Thoumine, Nerem et al. 1995; Yano, Geibel et al. 1997; Chien, Li et al. 1998; Civelekoglu, Tardy et al. 1998; Wittstein, Qiu et al. 2000; Shyy and Chien 2002; Kuebler, Uhlig et al. 2003; Mazzag, Tamaresis et al. 2003; Hirakawa, Oike et al. 2004; Li, Haga et al. 2005; Thamilselvan and Basson 2005; Kisucka, Butterfield et al. 2006; Lehoux, Castier et al. 2006).

Along with fluid shear stress (τ), defined by blood viscosity (η), laminar flow (Q), and the inverse proportion of the vessel radius (r), these mechanical forces may prove to be major factors in regulating vascular cell phenotype and vessel structure, possibly through the production of soluble mediators (Noris, Morigi et al. 1995; Ranjan, Xiao et al. 1995; Malek, Izumo et al. 1999; Nagel, Resnick et al. 1999; Morawietz, Talanow et al. 2000; Liang, Huang et al. 2002; Li, Zheng et al. 2004; Ganguli, Persson et al. 2005; Li, Zheng et al. 2005; Cicha, Goppelt-Struebe et al. 2007). The lack of prior studies linking alterations in flow to soluble mediators in end stage liver disease, including those related to angiogenesis, testifies to the immaturity of the art in this area. It is this lack of a single, unifying theory relating the peripheral vasodilatation hypothesis, Ohm's law, and the clinical observations of increased venous proliferation that has hampered the development of effective therapies to treat portal hypertension.

Despite these developments, there remains a need for effective therapies of end-stage liver disease.

### BRIEF SUMMARY

In accordance with the purpose of this invention, as embodied and broadly described herein, this invention relates to angiogenesis antagonists and methods of using the angiogenesis antagonists. For example, disclosed herein are methods of using of the humanized monoclonal antibody bevacizumab, and potentially other anti-angiogenic therapies, to abrogate the formation of portal-systemic collaterals and progression of disease in patients with end-stage liver disease. Also disclosed are methods of using of the humanized monoclonal antibody bevacizumab, and potentially other anti-angiogenic therapies, to alter sheer stress in a subject.

Inherent in this therapeutic application is the understanding that alterations in flow, pressure, and intraluminal shear stress lead to the elaboration of angiogenic factors in the venous circulation which, by extension, can also be used to invoke their use in other models of venous disease. Examples of such models include, but are not limited to, hemorrhoids, varicose veins, dialysis-associated venous hypertension, three diseases known to induce abnormal proliferation of veins in response to changes in pressure.

Also disclosed herein are uses of an angiogenesis antagonist in the preparation of a medicament for the treatment of end-stage liver disease in a subject.

Also disclosed are methods of treating a subject with end-stage liver disease comprising administering an effective amount of an angiogenesis antagonist to the subject.

Also disclosed are methods of treating a subject with end-stage liver disease comprising administering an effective amount of a pharmaceutical composition comprising an angiogenesis antagonist and a pharmaceutically acceptable carrier to the subject.

Also disclosed are methods of reducing end-stage liver disease complications in a subject comprising administering an effective amount of an angiogenesis antagonist to the subject.

Also disclosed are methods of reducing end-stage liver disease complications in a subject comprising administering an effective amount of a pharmaceutical composition comprising an angiogenesis antagonist and a pharmaceutically acceptable carrier to the subject.

Also disclosed are methods of preventing end-stage liver disease complications in a subject comprising administering an effective amount of an angiogenesis antagonist to the subject.

Also disclosed are methods of treating end-stage liver disease complications in a subject comprising administering an effective amount of an angiogenesis antagonist to the subject.

Also disclosed are methods of preventing the formation of portal-systemic collaterals in a subject comprising administering an effective amount of an angiogenesis antagonist to the subject.

Also disclosed are methods of treating or preventing acute or chronic liver inflammation in a subject, comprising administering to the subject an effective amount of a dithiocarbamate.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows peripheral vasodilatation hypothesis

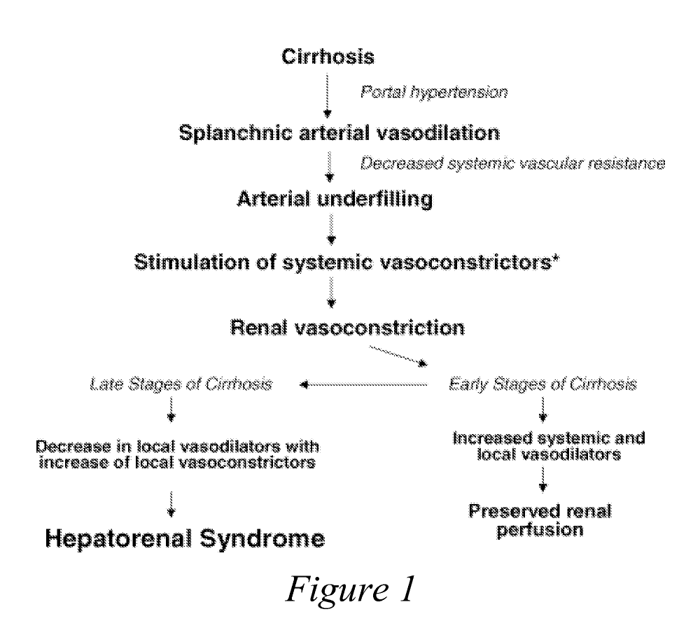

FIG. 2 shows progression of hepatorenal syndrome in the setting of the peripheral vasodilatation hypothesis.

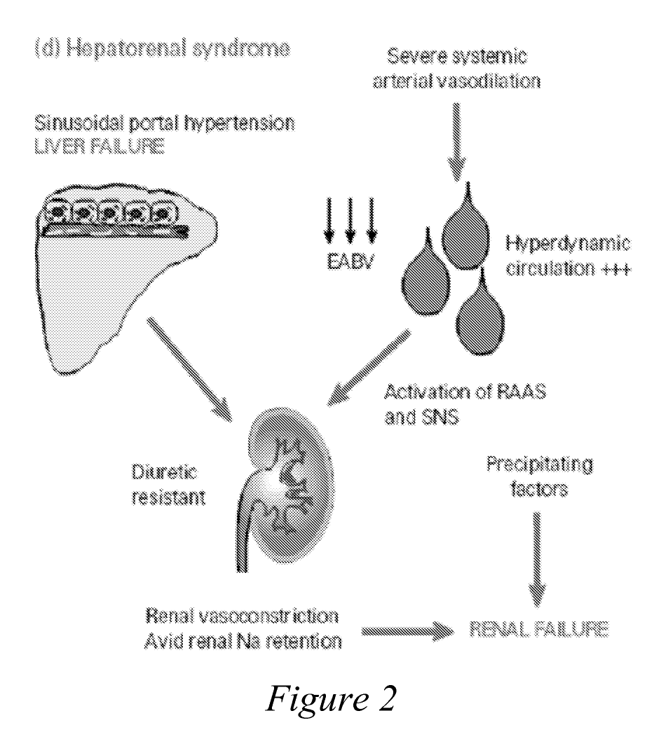

FIG. 3 shows intracellular signaling after binding of the VEGF molecule

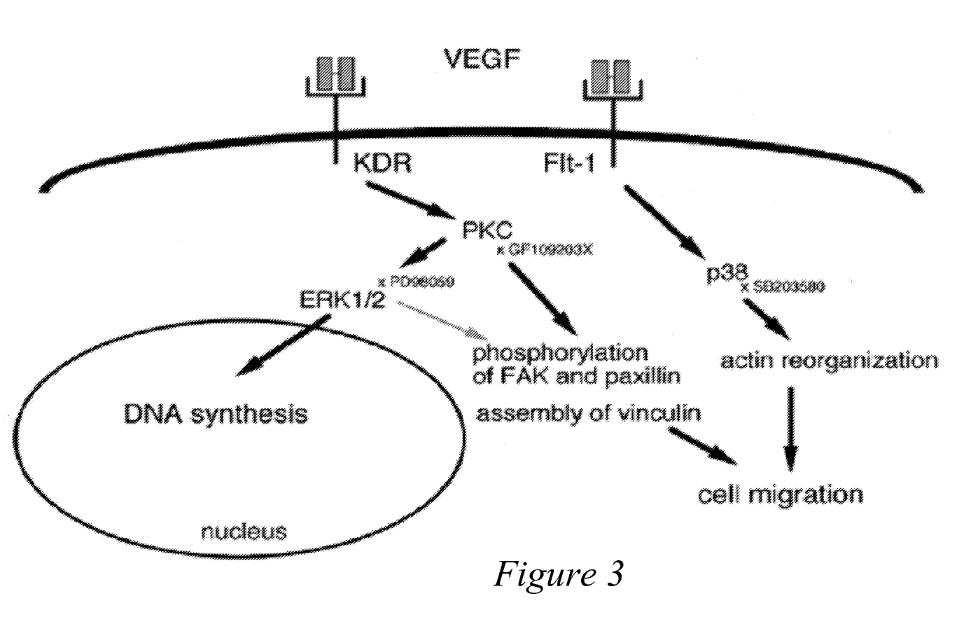

FIG. 4 shows downstream intracellular signaling and mechanisms of cellular proliferation involved with angiogenesis

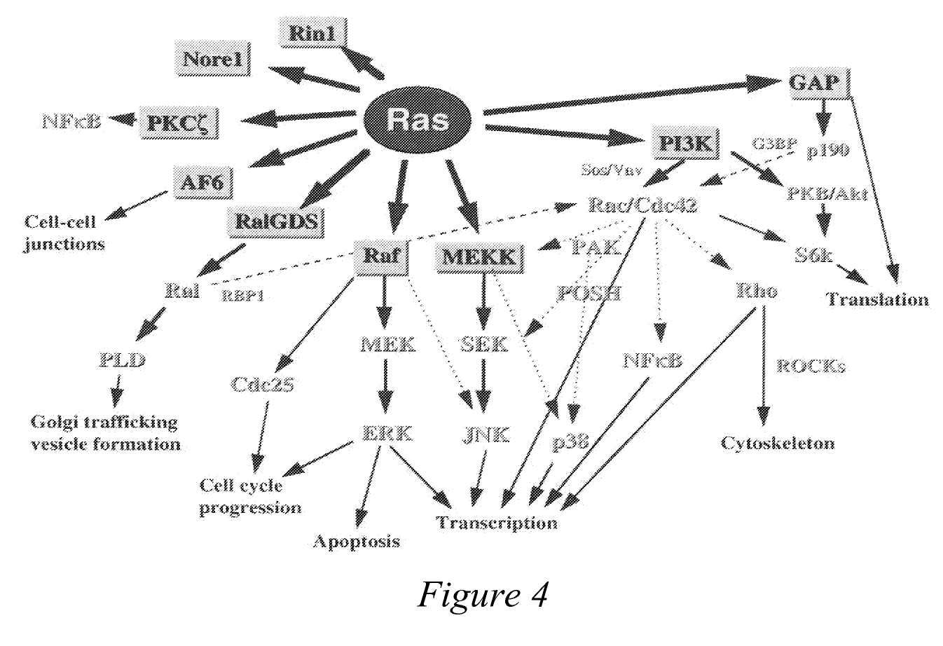

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. For example, it is understood that the disclosed method and compositions are not limited to specific synthetic methods, or to specific recombinant biotechnology methods unless otherwise specified, or to particular reagents unless otherwise specified, to specific pharmaceutical carriers, or to particular pharmaceutical formulations or administration regimens, as such may, of course, vary.

It is also to be understood that the terminology used herein is for the purpose of describing particular embodiments only, and is not intended to limit the scope of the present invention which will be limited only by the appended claims.

### A. COMPOSITIONS

Disclosed herein are compositions related to angiogenic and inflammatory factors. For example, disclosed herein are angiogenesis antagonists capable of blocking, inhibiting, abrogating, interfering or reducing pathological angiogenesis associated with end-stage liver disease and end-stage liver disease complication. Also disclosed herein are anti-inflammatory agents capable of blocking, inhibiting, abrogating, interfering or reducing acute and/or chronic inflammation associated with end-stage liver disease and end-stage liver disease complication. The embodiments described above and below are useful with any of the compositions and methods disclosed herein.

1. Angiogenesis Antagonists

Angiogenesis antagonists can be any composition, including nucleic acids, proteins, or antibodies, capable of blocking, inhibiting, abrogating, interfering or reducing pathological angiogenesis associated with a disease or disorder. The methods and articles of manufacture of the present invention use, or incorporate, an angiogenesis antagonist. Accordingly, disclosed herein are non-limiting examples of angiogenesis antagonists that can be used in the compositions and methods disclosed herein. It is understood, however, that the skilled artisan can select additional angiogenesis antagonists from those available in the art for use in the herein disclosed compositions and methods without undue experimentation.

In some aspect, the disclosed angiogenesis antagonists can inhibit an activity of vascular endothelial growth factor (VEGF). “Activities” of a protein include, for example, transcription, translation, intracellular translocation, secretion, phosphorylation by kinases, cleavage by proteases, homophilic and heterophilic binding to other proteins, ubiquitination. Thus, the disclosed angiogenesis antagonists can, for example, block the binding of VEGF to its receptor. For example, the disclosed angiogenesis antagonists can block the binding of VEGF-A to one or more of VEGFR-1 (Flt-1) and VEGFR-2 (Flk-1/KDR). In other aspects, the disclosed angiogenesis antagonists can, for example, inhibit the expression of VEGF, such as VEGF-A. Thus, the disclosed angiogenesis antagonists can inhibit transcriptional activation of VEGF expression, translation of VEGF, post-translational processing of VEGF, and/or secretion of VEGF from the cell. In some aspects, the disclosed angiogenesis antagonists can block the intracellular signal transduction involved in activating VEGF gene expression. For example, nuclear factor-κB (NF-κB) is involved in activating VEGF gene expression. In other aspects, the disclosed angiogenesis antagonists can block the intracellular signal transduction following VEGF activation of its receptor.

i. Nucleic Acid

The disclosed angiogenesis inhibitors can comprise a nucleic acid. Nucleic acids of the disclosed compositions and methods can be a functional nucleic acid. The nucleic acid of the disclosed compositions and methods can encode a protein that acts as an antiangiogenic antagonist, wherein the nucleic acid is operably linked to an expression control sequence.

The nucleic acids of the present invention are referred to herein interchangeably as angiogenesis antagonist nucleotides or angiogenesis antagonist polynucleotides. The disclosed nucleic acids are made up of for example, nucleotides, nucleotide analogs, or nucleotide substitutes. Non-limiting examples of these and other molecules are discussed herein. It is understood that for example, when a vector is expressed in a cell that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

The nucleotides of the invention can comprise one or more nucleotide analogs or substitutions. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (ψ), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH2)n O]m CH3, —O(CH2)n OCH3, —O(CH2)n NH2, —O(CH2)n CH3, —O(CH2)n —ONH2, and — (CH2)n ON[(CH2)n CH3)]2, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2 CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH2 and S.

Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety for their teaching of modifications and methods related to the same.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference in its entirety for their teaching of modifications and methods related to the same.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety or sugar moieties replaced. Nucleotide substitutes do not contain a standard phosphorus atom. Substitutes for the phosphate can be, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference in its entirety for their teaching of modifications and methods related to the same.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference in its entirety for their teaching of modifications and methods related to the same. (See also Nielsen et al., Science, 254, 1497-1500 (1991)).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference in its entirety for their teaching of modifications and methods related to the same.

The same methods of calculating homology as described elsewhere herein concerning polypeptides can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

Also disclosed are angiogenic antagonsists that are functional nucleic acids that can interact with angiogenic factors. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of polynucleotide sequences of angiogeneic factors or the genomic DNA of angiogeneic factors or they can interact with the polypeptide encoded by a angiogeneic factor polynucleotide sequences. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Also disclosed herein are antisense molecules that interact with angiogenic factor polynucleotides. Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (kd) less than or equal to 10-6, 10-8, 10-10, or 10-12. A representative sample of methods and techniques which aid in the design and use of antisense molecules can be found in the following non-limiting list of United States patents: U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437 each of which is herein incorporated by reference in its entirety for their teaching of modifications and methods related to the same.

Also disclosed are aptamers that interact with angiogenic factor polynucleotides. For example, disclosed are aptamers capable of blocking VEGF or VEGFR

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kds from the target molecule of less than 10-12 M. It is preferred that the aptamers bind the target molecule with a kd less than 10-6, 10-8, 10-10, or 10-12. Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a kd with the target molecule at least 10, 100, 1000, 10,000, or 100,000 fold lower than the kd with a background binding molecule. It is preferred when doing the comparison for a polypeptide for example, that the background molecule be a different polypeptide. For example, when determining the specificity of aptamers, the background protein could be ef-1α. Representative examples of how to make and use aptamers to bind a variety of different target molecules can be found in the following non-limiting list of United States patents: U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Also disclosed are ribozymes that interact with the angiogenic factor polynucleotides. Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following United States patents: U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following United States patents: U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following United States patents: U.S. Pat. Nos. 5,595,873 and 5,652,107). There are also a number of ribozymes that are not found in natural systems, but which have been engineered to catalyze specific reactions de novo (for example, but not limited to the following United States patents: U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of United States patents: U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Also disclosed are triplex forming functional nucleic acid molecules that interact with angiogenic factor polynucleotides. Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a kd less than 10-6, 10-8, 10-10, or 10-12. Representative examples of how to make and use triplex forming molecules to bind a variety of different target molecules can be found in the following non-limiting list of United States patents: U.S. Pat. Nos. 5,176,996, 5,645,985, 5,650,316, 5,683,874, 5,693,773, 5,834,185, 5,869,246, 5,874,566, and 5,962,426.

Also disclosed are external guide sequences that can form a complex with angiogenic factor polynucleotides. External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules can be found in the following non-limiting list of United States patents: U.S. Pat. Nos. 5,168,053, 5,624,824, 5,683,873, 5,728,521, 5,869,248, and 5,877,162.

Also disclosed are polynucleotides that contain peptide nucleic acids (PNAs) compositions. PNA is a DNA mimic in which the nucleobases are attached to a pseudopeptide backbone (Good and Nielsen, Antisense Nucleic Acid Drug Dev. 1997; 7(4) 431-37). PNA is able to be utilized in a number of methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol 1997 June; 15(6):224-9). As such, in certain embodiments, one may prepare PNA sequences that are complementary to one or more portions of an mRNA sequence based on the disclosed polynucleotides, and such PNA compositions may be used to regulate, alter, decrease, or reduce the translation of the disclosed polynucleotides transcribed mRNA, and thereby alter the level of the disclosed polynucleotide's activity in a host cell to which such PNA compositions have been administered.

PNAs have 2-aminoethyl-glycine linkages replacing the normal phosphodiester backbone of DNA (Nielsen et al., Science Dec. 6, 1991; 254(5037):1497-500; Hanvey et al., Science. Nov. 27, 1992; 258(5087):1481-5; Hyrup and Nielsen, Bioorg Med. Chem. 1996 January; 4(1):5-23). This chemistry has three important consequences: firstly, in contrast to DNA or phosphorothioate oligonucleotides, PNAs are neutral molecules; secondly, PNAs are achirial, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med. Chem. 1995 April; 3(4):437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med. Chem. 1995 April; 3(4):437-45; Petersen et al., J Pept Sci. 1995 May-June; 1(3):175-83; Orum et al., Biotechniques. 1995 September; 19(3):472-80; Footer et al., Biochemistry. Aug. 20, 1996; 35(33): 10673-9; Griffith et al., Nucleic Acids Res. Aug. 11, 1995; 23(15):3003-8; Pardridge et al., Proc Natl Acad Sci USA. Jun. 6, 1995; 92(12):5592-6; Boffa et al., Proc Natl Acad Sci USA. Mar. 14, 1995; 92(6):1901-5; Gambacorti-Passerini et al., Blood. Aug. 15, 1996; 88(4):1411-7; Armitage et al., Proc Natl Acad Sci USA. Nov. 11, 1997; 94(23):12320-5; Seeger et al., Biotechniques. 1997 September; 23(3):512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. Dec. 15, 1993; 65(24):3545-9) and Jensen et al. (Biochemistry. Apr. 22, 1997; 36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

Also disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polypeptide is disclosed and discussed and a number of modifications that can be made to a number of molecules including the polypeptide are discussed, specifically contemplated is each and every combination and permutation of polypeptide and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

One way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference in its entirety and at least for material related to hybridization of nucleic acids). As used herein “stringent hybridization” for a DNA:DNA hybridization is about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their kd, or where only one of the nucleic acid molecules is 10 fold or 100 fold or 1000 fold or where one or both nucleic acid molecules are above their kd.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein. Optionally, one or more of the isolated polynucleotides of the invention are attached to a solid support. Solid supports are disclosed herein.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

In addition, the disclosed polynucleotides can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood, to a target organ, or inhaled into the respiratory tract to target cells of the respiratory tract. For example, a composition comprising a polynucleotide described herein and a cationic liposome can be administered to a subjects lung cells. Regarding liposomes, see, e.g., Brigham et al. Am. J. Resp. Cell. Mol. Biol. 1:95-100 (1989); Feigner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described herein, delivery of the compositions to cells can be via a variety of mechanisms. As one example, delivery can be via a liposome, using commercially available liposome preparations such as LIPOFECTIN™, LIPOFECTAMINE™ (GIBCO-BRL, Gaithersburg, Md.), SUPERFECT™ (Qiagen, Hilden, Germany) and TRANSFECTAM™ (Promega Biotec, Madison, Wis.), as well as other liposomes developed according to procedures standard in the art. In addition, the disclosed nucleic acid or vector can be delivered in vivo by electroporation, the technology for which is available from Genetronics (San Diego, Calif.) as well as by means of a SONOPORATION™ machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

As described herein, the compositions can be administered in a pharmaceutically acceptable carrier and can be delivered to the subject's cells in vivo or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

The nucleic acids, such as, the polynucleotides described herein, can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 3rd Edition (Cold Spring Harbor Laboratory Press, Cold Spring Harbor, N.Y., 2001) Chapters 5, 6) to purely synthetic methods, for example, by the cyanoethyl phosphoramidite method using a Milligen or Beckman System 1Plus DNA synthesizer. Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

ii. Protein

The angiogenesis antagonist can also be a protein antagonist of an angiogenic factor. For example, the angiogenesis antagonist can be a VEGF variant or soluble VEGF receptor capable of binding VEGF receptor or VEGF, respectively, with reduced or no cellular VEGF receptor activation. The angiogenesis can also be small peptide linked to an internalization sequence, wherein the peptide blocks a protein involved in activation of VEGF expression. For example, the peptide can bind and/or inhibit NFκB activity. Other such examples

As used herein, the term “polypeptide” is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response.

The polypeptides of the present invention are sometimes herein referred to as a angiogenesis antagonist proteins or angiogenesis antagonist polypeptides, as an indication that their identification has been based at least in part upon their expression in cancer samples isolated from tissues of a subject with lung cancer, head and neck cancer, or melanoma. The peptides described herein are identified from tissues for a subject with either lung cancer, head and neck cancer, or melanoma. Accordingly, such a peptide may not be present in adjacent normal tissue.

Additionally, polypeptides described herein may be identified by their different reactivity with sera from subjects with end-stage liver disease or subjects with end-stage liver disease complications as compared to sera from unaffected individuals. For example, polypeptides described herein may be identified by their reactivity with sera from subjects with a end-stage liver disease as compared to their lack of reactivity to sera from unaffected individuals. Additionally, polypeptides described herein may be identified by their reactivity with sera from subjects with end-stage liver disease as compared to their higher reactivity to sera from unaffected individuals. Additionally, polypeptides described herein may be identified by their reactivity with sera from subjects with a end-stage liver disease as compared to their lower reactivity to sera from unaffected individuals.

Also disclosed are isolated angiogenesis antagonist polypeptides with substituted, inserted or deletional variations. Insertions include amino or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion.

Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture.

Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

Substantial changes in function or immunological identity are made by selecting substitutions that are less conservative than those in Tables 1 and 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation or glycosylation.

TABLE 1
Amino Acid Abbreviations

| Amino Acid | Abbreviations | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

For example, the replacement of one amino acid residue with another that is biologically and chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

It is understood that the description of conservative mutations and homology can be combined together in any combination, such as embodiments that have at least 70% homology to a particular sequence wherein the variants are conservative mutations.

It is understood that there are numerous amino acid and peptide analogs which can be incorporated into the disclosed compositions. For example, there are numerous D amino acids or amino acids which have a different functional substituent then the amino acids shown in Tables 1 and 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH₂H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

iii. Antibody

As described above, the angiogenesis antagonist can be an antibody, an antibody fragment or an antigen-binding fragment thereof. For example, the angiogenesis antagonist can be an anti-VEGF antibody or a neutralizing anti-VEGFR antibody capable of blocking VEGF binding to VEGFR.

Optionally, the isolated antibodies, antibody fragments, or antigen-binding fragment thereof can be neutralizing antibodies. The antibodies, antibody fragments and antigen-binding fragments thereof disclosed herein can be identified using the methods disclosed herein. For example, antibodies that bind to the polypeptides of the invention can be isolated using the antigen microarray described above.

The anti-VEGF antibody “Bevacizumab (BV)”, also known as “rhuMAb VEGF” or “Avastin®”, is a recombinant humanized anti-VEGF monoclonal antibody generated according to Presta et al. (1997) Cancer Res. 57:4593-4599. It comprises mutated human IgG1 framework regions and antigen-binding complementarity-determining regions from the murine anti-hVEGF monoclonal antibody A4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Thus, the anti-VEGF antibody can be a monoclonal antibody that binds to the same epitope as the monoclonal anti-VEGF antibody A4.6.1 produced by hybridoma ATCC HB 10709. Thus, the anti-VEGF antibody can be a recombinant humanized anti-VEGF monoclonal antibody generated according to Presta et al. (1997) Cancer Res. 57:4593-4599, including but not limited to the antibody known as bevacizumab (BV; Avastin®).

The term “antibody” or “antibodies” is used herein in a broad sense and includes both polyclonal and monoclonal antibodies. In addition to intact immunoglobulin molecules, also disclosed are antibody fragments or polymers of those immunoglobulin molecules, and human or humanized versions of immunoglobulin molecules or fragments thereof, as long as they are chosen for their ability to interact with the polypeptides disclosed herein.

“Antibody fragments” are portions of a complete or native antibody, preferably comprising the antigen-binding or variable region thereof. A complete antibody refers to an antibody having two complete light chains and two complete heavy chains. An antibody fragment lacks all or a portion of one or more of the chains. Examples of antibody fragments include, but are not limited to, half antibodies and fragments of half antibodies. A half antibody is composed of a single light chain and a single heavy chain. Half antibodies and half antibody fragments can be produced by reducing an antibody or antibody fragment having two light chains and two heavy chains. Such antibody fragments are referred to as reduced antibodies. Reduced antibodies have exposed and reactive sulfhydryl groups. These sulfhydryl groups can be used as reactive chemical groups or coupling of biomolecules to the antibody fragment. Examples of antibody fragments include Fab, Fab′, F(ab′)2, and Fv fragments; diabodies; linear antibodies; single-chain antibody molecules; and multispecific antibodies formed from antibody fragments. The hinge region of an antibody or antibody fragment is the region where the light chain ends and the heavy chain goes on.

Antibody fragments for use in antibody conjugates can bind antigens. Preferably, the antibody fragment is specific for an antigen. An antibody or antibody fragment is specific for an antigen if it binds with significantly greater affinity to one epitope than to other epitopes. The antigen can be any molecule, compound, composition, or portion thereof to which an antibody fragment can bind. An analyte can be any molecule, compound or composition of interest. For example, the antigen can be a polynucleotide of the invention.

The antibodies or antibody fragments can be tested for their desired activity using the in vitro assays described herein, or by analogous methods, after which their in vivo therapeutic or prophylactic activities are tested according to known clinical testing methods.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a .beta.-sheet configuration, connected by three hypervariable regions, which form loops connecting, and in some cases forming part of, the .beta.-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, Δ, ε, γ, and μ, respectively. The subunit structures and three-dimensional configurations of different classes of immunoglobulins are well known.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. For a review of scFv see Pluickthun in The Pharmacology of Monoclonal Antibodies, vol. 113, Rosenburg and Moore eds., Springer-Verlag, N.Y., pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A description follows as to exemplary techniques for the production of the antibody antagonists used in accordance with the present invention.

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl2, or R1N═C=NR, where R and R1 are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The disclosed monoclonal antibodies can also by obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol, 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies can also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, such as an Fv, Fab, Fab′, or other antigen-binding portion of an antibody, can be accomplished using routine techniques known in the art. For instance, digestion can be performed using papain. Examples of papain digestion are described in WO 94/29348 published Dec. 22, 1994 and U.S. Pat. No. 4,342,566 which is hereby incorporated by reference in its entirety for its teaching of papain digestion of antibodies to prepare monovaltent antibodies. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody which are responsible for antigen-binding. The hypervariable region comprises amino acid residues from a “complementarity determining region” or “CDR” (e.g. residues 24-34 (L1), 50-56 (L2) and 89-97 (L3) in the light chain variable domain and, 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

Optionally, the disclosed human antibodies can be made from memory B cells using a method for Epstein-Barr virus transformation of human B cells. (See, e.g., Triaggiai et al., An efficient method to make human monoclonal antibodies from memory B cells: potent neutralization of SARS coronavirus, Nat. Med. 2004 August; 10(8):871-5. (2004)), which is herein incorporated by reference in its entirety for its teaching of a method to make human monoclonal antibodies from memory B cells). In short, memory B cells from a subject who has survived a natural infection are isolated and immortalized with EBV in the presence of irradiated mononuclear cells and a CpG oligonucleotide that acts as a polyclonal activator of memory B cells. The memory B cells are cultured and analyzed for the presence of specific antibodies. EBV-B cells from the culture producing the antibodies of the desired specificity are then cloned by limiting dilution in the presence of irradiated mononuclear cells, with the addition of CpG 2006 to increase cloning efficiency, and cultured. After culture of the EBV-B cells, monoclonal antibodies can be isolated. Such a method offers (1) antibodies that are produced by immortalization of memory B lymphocytes which are stable over a lifetime and can easily be isolated from peripheral blood and (2) the antibodies isolated from a primed natural host who has survived a natural infection, thus eliminating the need for immunization of experimental animals, which may show different susceptibility and, therefore, different immune responses.

Other methods for humanizing non-human antibodies have also been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol, 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol.

222:581-597 (1991), or Griffith et al., EMBO J. 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fa′-SH fragments can be directly recovered from E. coli and chemically coupled to form F(ab′)2 fragments (Carter et al., Bio/Technology 10:163-167 (1992)). According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

Bispecific antibodies are antibodies that have binding specificities for at least two different epitopes. Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986). According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

The antibodies disclosed herein can also be administered to a subject. Nucleic acid approaches for antibody delivery also exist. The broadly neutralizing antibodies to the polypeptides disclosed herein and antibody fragments can also be administered to subjects or subjects as a nucleic acid preparation (e.g., DNA or RNA) that encodes the antibody or antibody fragment, such that the subject's own cells take up the nucleic acid and produce and secrete the encoded antibody or antibody fragment.

2. Dithiocarbamates

As disclosed herein, dithiocarbamates can inhibit angiogenesis and liver inflammation. Thus, disclosed herein are compositions comprising dithiocarbamate. Dithiocarbamates are a broad class of molecules that have the ability to chelate metal ions, as well as react with sulfhydryl groups and glutathione. After metal-mediated conversion to their corresponding disulfides, dithiocarbamates inhibit cysteine proteases by forming mixed disulfides with critical protein thiols. Thus, in some aspects, the dithiocarbamate of the disclosed compositions and methods is in a reduced thioacid form.

In addition to their reduced thioacid form, dithiocarbamates can also or are known to exist in four other forms: a) the disulfide, a condensed dimer of the thioacid with elimination of reduced sulfhydryl groups by disulfide bond formation; b) the negatively charged thiolate anion, generally as a salt, such as the sodium salt or ammonium salt; c) the 1,1-dithiolato coordination complex of metal ions in which the two adjoining sulfur atoms of the dithiocarbamate are bound to the same metal ion, for example, titanium(III), vanadium(III), chromium(III), iron(III), cobalt(III), nickel(II), copper(II), silver(I), gold(III), Zn(II), Au(I), Mn(III), Ga(III), Pt(II); and d) the monodentate dithiolato coordination complex in which either one of the sulfur atoms binds to a metal ion, for example titanium(III), vanadium(III), chromium(III), iron(III), cobalt(III), nickel(II), copper(II), silver(I), or gold(III). The disulfide, thiolate anion, and coordination complexes of dithiocarbamates are all structurally distinct from the reduced form of PDTC used by Chinery, et al., in that they have no reduced sulfhydryl molecular moiety and are incapable of functioning as antioxidants by donating the proton from a reduced sulfhydryl to scavenge electrons of free radical species.

Thus, in some aspects, the dithiocarbamate of the disclosed compositions and methods is a dithiocarbamate disulfide. Thus, in some aspects, the angiogenesis inhibitor of the disclosed compositions and methods is a thiolate anion. Thus, in some aspects, the dithiocarbamate of the disclosed compositions and methods is a coordination complex.

In some aspects, the dithiocarbamates of the disclosed compositions and methods can be identified and/or selected based on its ability to block nuclear factor-KB (NF-KB), inhibit VEGF expression, and/or inhibit angiogenesis.

In some aspects, the dithiocarbamate of the disclosed compositions and methods is a dithiocarbamate thiolate anion. As is known in the art, dithiocarbamates react with critical thiols and also complex metal ions. Thus, the dithiocarbamate of the disclosed compositions and methods can be a coordination compound.

However, dithiocarbamates and metal ions can have deleterious effects when co-administrated. Thus, in other aspects, the dithiocarbamate of the disclosed compositions and methods is dithiocarbamate which is separately administered to a subject with a metal ion. In some aspects, the separately administered dithiocarbamates and metal ions accumulate in the liver and there form a coordination compound.

A therapeutically effect amount of the herein disclosed dithiocarbamate anion compound and an intracellular metal ion stimulant, which can enhance the intracellular level of the above described metal ions in the liver of the subject, can therefore be separately administered to a subject. Intracellular heavy metal ion carriers are known. For example, ceruloplasmin can be administered to the patient to enhance the intracellular copper level. Other metal ion carriers known in the art may also be administered in accordance with this aspect of the invention. The heavy metal ion carriers and the dithiocarbamate disulfide or metal anion can be administered together or separately.

Ceruloplasmin is a protein naturally produced by the human body and can be purified from human serum. This 132-kD glycoprotein, which carries 7 copper(II) ions complexed over three 43-45 kD domains, is an acute phase reactant and the major copper-carrying protein in human plasma. See Halliwell, et al., Methods Enzymol. 186:1-85 (1990). When transported into cells, at least some of the bound copper(II) ions can be accessible for complexation with the dithiocarbamate disulfide or thiolate anion administered to the patient. (See Percival, et al., Am. J. Physiol. 258:3140-3146 (1990).) Ceruloplasmin and dithiocarbamate disulfides or thiolate anions are typically administered in different compositions. Dithiocarbamate disulfides or thiolate anions can be administered at about the same time, or at some time apart. For example, ceruloplasmin can be administered from about five minutes to about 12 hours before or after dithiocarbamate disulfide or thiolate anions are administered to the patient.

In some aspects the dithiocarbamate is disulfuram. For example, disulfuram can be separately administered with a heavy metal ion to a subject at currently approved doses for alcoholism. The metal ion can be coordinated to a chelating agent such as acetate, lactonate, glycinate, citrate, propionate, or gluconate, with a pharmaceutically acceptable counter ion. In some aspects, the heavy metal ion is zinc. Thus, in some aspects, disulfuram is separately administered with chelated zinc (e.g., zinc gluconate) to the subject.

In some aspects, zinc is administered first, and disulfuram is administered after a time sufficient for a substantial amount of the zinc to have passed out of the gastrointestinal system into the blood stream.

Disulfuram and its diethyldithiocarbamate anion are effective when administered at amounts within the conventional clinical ranges determined in the art. Disulfuram approved by the U.S. Food and Drug administration (Antabuse™) can be purchased in 250 and 500 mg tablets for oral administration from Odyssey Pharmaceuticals, East Hanover, N.J. 07936. Typically, it is effective at an amount of from about 125 to about 1000 mg per day, preferably from 250 to about 500 mg per day for disulfuram and 100 to 500 mg per day or 5 mg/kg intravenously or 10 mg/kg orally once a week for diethyldithiocarbamate. However, the dosage can vary with the body weight of the patient treated. The active ingredient may be administered at once, or may be divided into a number of smaller doses to be administered at predetermined intervals of time. The suitable dosage unit for each administration of disulfuram is, e.g., from about 50 to about 1000 mg/day, preferably from about 250 to about 500 mg/day. The desirable peak concentration of disulfuram generally is about 0.05 to about 10 μM, preferably about 0.5 to about 5 μM, in order to achieve a detectable therapeutic effect. Similar concentration ranges are desirable for dithiocarbamate thiolate anions and for dithiocarbamate-metal ion chelate compounds.

Disulfuram implanted subcutaneously for sustained release has also been shown to be effective for alcoholism at an amount of 800 to 1600 mg to achieve a suitable plasma concentration. This can be accomplished by using aseptic techniques to surgically implant disulfuram into the subcutaneous space of the anterior abdominal wall. (See e.g., Wilson, et al., J. Clin. Psych. 45:242-247 (1984).) In addition, sustained release dosage formulations, such as an 80% poly(glycolic-co-L-lactic acid) and 20% disulfuram, can be used. The therapeutically effective amount for other dithiocarbamate disulfide compounds can also be estimated or calculated based on the above dosage ranges of disulfuram and the molecular weights of disulfuram and the other dithiocarbamate disulfide compound, or by other methods known in the art.

Minimal side effects on this dosage regimen include a metallic taste in the mouth, flatulence, and intolerance to alcoholic beverages. An enteric-coated oral dosage form of diethyldithiocarbamate anions to liberate active drug only in the alkaline environment of the intestine is preferred because of the potential for liberation of carbon disulfide upon exposure of diethyldithiocarbamate to hydrochloric acid in the stomach. An oral enteric-coated form of sodium diethyldithiocarbamate is available in 125 mg tablets as Imuthiol® through Institute Merieux, Lyon, France.

Metal ions can be administered separately as aqueous solutions. In the case of charged metal ion coordination complexes, the metal ions can be administered in a pharmaceutically suitable form. Ideally, the charged metal species contains the metal ion coordinated to a chelating agent such as acetate, lactonate, glycinate, citrate, propionate, or gluconate, with a pharmaceutically acceptable counter ion. In some aspects, the amount of metal ion to be used is proportional to the amount of dithiocarbamate to be administered based on the stoichiometric ratio between a metal ion and the dithiocarbamate.

3. Zinc

Also disclosed herein is a composition comprising zinc or a pharmaceutically acceptable salt or chelate thereof (e.g., zinc gluconate, zinc acetate, zinc sulfate, or zinc chloride). Zinc plays a critical role in cellular biology, and is involved in virtually every important cellular process such as transcription, translation, ion transport, and apoptosis. However, as all eukaryotic cells strictly regulate the membrane transport of Zn²⁺, making it very difficult to modulate the intracellular concentration and distribution of Zn²⁺, the disclosed composition can comprise zinc that is separately administered to a subject with a zinc ionophore. The zinc ionophore can facilitate the transport of Zn²⁺ into the target cells. An example of a zinc ionophore is zinc pyrithione (zinc pyridinethione, C₁₀H₈N₂O₂S₂Zn, MW 317.75), which is the active ingredient in anti-dandruff shampoo (U.S. Pat. Nos. 3,236,733 and 3,281,366) as well as a number of other topical skin treatment formulations. It is a fungicide and bactericide at high concentrations. It is highly lipophilic and therefore penetrates membranes easily. This permits zinc pyrithione to transport zinc across cell membranes, thereby conferring on zinc pyrithione the properties of a zinc ionophore. However, zinc pyrithione is toxic when ingested. Thus, in some aspects, the zinc ionophore is not zinc pyrithione.

Thus, the a zinc ionophore of the disclosed composition can be any non-toxic compound capable of binding zinc with moderate affinity and having sufficient lipophilic properties to penetrate cell membranes. Thus, zinc ionophore can be a dithiocarbamate. Non-limiting examples of dithiocarbamates include pyrrolidine dithiocarbamate, diethyldithiocarbamate, disulfuram, and dimethyldithiocarbamate.

Zinc can be administered separately as an aqueous solution. In the case of charged zinc ion coordination complexes, the zinc ions can be administered in a pharmaceutically suitable form. Ideally, the zinc ions are coordinated to a chelating agent such as acetate, lactonate, glycinate, citrate, propionate, or gluconate, with a pharmaceutically acceptable counter ion. In some aspects, the amount of zinc ion to be used is proportional to the amount of dithiocarbamate to be administered based on the stoichiometric ratio between a metal ion and the dithiocarbamate.

In some aspects, zinc is administered to the subject first, and disulfuram is administered to the subject after a time sufficient for a substantial amount of the zinc to have passed out of the gastrointestinal system and into the blood stream.

4. Copper Antagonist

Also disclosed herein is a composition comprising a copper antagonist. For example, the disclosed composition can comprise a copper chelator. In some aspects, the copper antagonist is penicillamine or trientine.

The steps required for successful tumor angiogenesis at the primary and metastatic sites are diverse, and they depend on an imbalance between angiogenesis activators such as vascular endothelial growth factor and basic fibroblast growth factor and inhibitors such as thrombospondin 1, angiostatin, and endostatin. The relative importance of the different angiogenesis-modulating molecules in different tissues can determine the relative potency of antiangiogenic compounds to elicit a response at both the primary and metastatic sites. Therefore, it is desirable that the antiangiogenic strategy affect multiple activators of angiogenesis. Because copper is a required cofactor for the function of many key mediators of angiogenesis, such as basic fibroblast growth factor, vascular endothelial growth factor, and angiogenin, modulation of total body copper status can be used as an antiangiogenic strategy for the treatment of cancer. One of the drugs currently being used as a new anticopper therapy for Wilson's disease, tetrathiomolybdate, shows unique and desirable properties of fast action, copper specificity, and low toxicity, as well as a unique mechanism of action. Tetrathiomolybdate forms a stable tripartite complex with copper and protein. If given with food, it complexes food copper with food protein and prevents absorption of copper from the GI tract. There is endogenous secretion of copper in saliva and gastric secretions associated with food intake, and this copper is also complexed by tetrathiomolybdate when it is taken with meals, thereby preventing copper reabsorption. Thus, patients are placed in a negative copper balance immediately when tetrathiomolybdate is given with food. If tetrathiomolybdate is given between meals, it is absorbed into the blood stream, where it complexes either free or loosely bound copper with serum albumin. This tetrathiomolybdate-bound copper fraction is no longer available for cellular uptake, has no known biological activity, and is slowly cleared in bile and urine.

Thus, in some aspects, the copper antagonist is tetrathiomolybdate (TM) or a pharmaceutically acceptable salt or chelate thereof. For example, the copper antagonist can be ammonium tetrathiomolybdate. Ammonium tetrathiomolybdate has the formula [NH₄]₂[MoS₄]. The thiometallate anion has the distinctive property of undergoing oxidation at the sulfur centers concomitant with reduction of the metal from Mo(VI) to Mo(IV). The salt contains the tetrahedral [MoS₄]²⁻ anion. The compound is prepared by treating solutions of molybdate, [MoO₄]²⁻ with hydrogen sulfide in the presence of ammonia:

[NH₄]₂[MoO₄]+4H₂S→[NH₄]₂[MoS₄]+4H₂O

Ammonium tetrathiomolybdate is a complex of sulfur and molybdenum designed as a fast-acting compound to quickly lower copper levels by oral chelation. This compound may be the world's safest and most potent anti-copper agent. It is extremely well tolerated, with few side effects, and ammonium tetrathiomolybdate is particularly useful to patients who wish to avoid the potential adverse reactions to the standard chelating agents, penicillamine and trientine.

5. Conjugations

The compositions used in the methods or included in the articles of manufacture herein can also be conjugated to a toxic agent to destroy neovascularization expressing VEGF or the VEGF receptor or induce apoptosis. For example the disclosed compositions could be conjugated to ricin.

The disclosed compositions used in the methods or included in the articles of manufacture herein can also be conjugated to an agent that activates the known apoptotic pathways. For example the angiogenesis antagonist could be conjgated to bcl-x.

The disclosed compositions used in the methods or included in the articles of manufacture herein can also be conjugated to a tyrosine kinase inhibitor. For example the disclosed compositions could be conjgated to sorafenib, sunitinib, AZD2171, Dasatinib, Erlotinib, Gefinitib, Imatinib, Lapatinib, Nilotinib, Semaxinib, or Vandetanib.

The disclosed compositions used in the methods or included in the articles of manufacture herein can also be conjugated to protein kinase C inhibitor or an inhibitor that is capable of inhibiting one or more of Ras, Rac, Rho, ERK, JNK, Akt, Raf, NF-kB, Cdc42.

Other modifications of the disclosed compositions are contemplated herein. For example, the disclosed compositions may be linked to one of a variety of nonproteinaceous polymers, e.g., polyethylene glycol, polypropylene glycol, polyoxyalkylenes, or copolymers of polyethylene glycol and polypropylene glycol.

6. Modifications

Amino acid sequence modification(s) of protein or peptide antagonists described herein are contemplated and described elsewhere herein. For example, it may be desirable to improve the binding affinity and/or other biological properties of the antagonist. Amino acid sequence variants of the antagonist are prepared by introducing appropriate nucleotide changes into the antagonist nucleic acid, or by peptide synthesis. Such modifications include, for example, deletions from, and/or insertions into and/or substitutions of, residues within the amino acid sequences of the antagonist. Any combination of deletion, insertion, and substitution is made to arrive at the final construct, provided that the final construct possesses the desired characteristics. The amino acid changes also may alter post-translational processes of the antagonist, such as changing the number or position of glycosylation sites.

A useful method for identification of certain residues or regions of the antagonist that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antagonist variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antagonist with an N-terminal methionyl residue or the antagonist fused to a cytotoxic polypeptide. Other insertional variants of the antagonist molecule include the fusion to the N- or C-terminus of the antagonist of an enzyme, or a polypeptide which increases the serum half-life of the antagonist.

Another type of variant is an amino acid substitution variant. These variants have at least one amino acid residue in the antagonist molecule replaced by different residue. The sites of greatest interest for substitutional mutagenesis of antibody antagonists include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened. TABLE-US-00001 TABLE 1 Original Exemplary Preferred Residue Substitutions Ala (A) val; leu; ile val Arg (R) lys; gln; asn lys Asn (N) gln; his; asp, lys; arg gln Asp (D) glu; asn glu Cys (C) ser; ala ser Gln (O) asn; glu asn Glu (E) asp; gln asp Gly (G) ala ala His (H) asn; gln; lys; arg Ile (I) leu; val; met; ala; leu phe; norleucine Leu (L) norleucine; ile; val; ile met; ala; phe Lys (K) arg; gln; asn arg Met (M) leu; phe; ile leu Phe (F) leu; val; ile; ala; tyr tyr Pro (P) ala ala Ser (S) thr thr Thr (T) ser ser Trp (W) tyr; phe tyr Tyr (Y) tip; phe; thr; ser phe Val (V) ile; leu; met; phe; leu ala; norleucine

Substantial modifications in the biological properties of the antagonist are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: [0125] (1) hydrophobic: norleucine, met, ala, val, leu, ile; [0126] (2) neutral hydrophilic: cys, ser, thr; [0127] (3) acidic: asp, glu; [0128] (4) basic: asn, gin, his, lys, arg; [0129] (5) residues that influence chain orientation: gly, pro; and [0130] (6) aromatic: trp, tyr, phe.

Non-conservative substitutions will entail exchanging a member of one of these classes for another class.

Any cysteine residue not involved in maintaining the proper conformation of the antagonist also may be substituted, generally with serine, to improve the oxidative stability of the molecule and prevent aberrant crosslinking Conversely, cysteine bond(s) may be added to the antagonist to improve its stability (particularly where the antagonist is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antagonist alters the original glycosylation pattern of the antagonist. By altering is meant deleting one or more carbohydrate moieties found in the antagonist, and/or adding one or more glycosylation sites that are not present in the antagonist.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antagonist is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antagonist (for O-linked glycosylation sites).

Nucleic acid molecules encoding amino acid sequence variants of the antagonist are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antagonist.

It may be desirable to modify the antagonist of the invention with respect to effector function, e.g. so as to enhance antigen-dependent cell-mediated cyotoxicity (ADCC) and/or complement dependent cytotoxicity (CDC) of the antagonist. This may be achieved by introducing one or more amino acid substitutions in an Fc region of an antibody antagonist. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989). To increase the serum half life of the antagonist, one may incorporate a salvage receptor binding epitope into the antagonist (especially an antibody fragment) as described in U.S. Pat. No. 5,739,277, for example. As used herein, the term “salvage receptor binding epitope” refers to an epitope of the Fc region of an IgG molecule (e.g., IgG.sub.1, IgG.sub.2, IgG.sub.3, or IgG.sub.4) that is responsible for increasing the in vivo serum half-life of the IgG molecule.

7. Therapeutic Formulations

Therapeutic formulations of the disclosed compositions used in accordance with the present invention are prepared for storage by mixing an angiogenesis antagonist having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN.™, PLURONICS.™ or polyethylene glycol (PEG).

Lyophilized formulations adapted for subcutaneous administration are described in WO97/04801. Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine or immunosuppressive agent (e.g. one which acts on T cells, such as cyclosporin or an antibody that binds T cells, e.g. one which binds LFA-1). The effective amount of such other agents depends on the amount of antagonist present in the formulation, the type of disease or disorder or treatment, and other factors discussed above. These are generally used in the same dosages and with administration routes as used hereinbefore or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This can be readily accomplished by filtration through sterile filtration membranes.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)), all of which are herein incorporated by reference in their entirety for their teaching of the same. Vehicles such as “stealth” and other antibody conjugated liposomes, drugs, receptor mediated targeting of DNA through cell specific ligands, and lymphocyte directed targeting. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. For example, the disclosed angiogenesis antagonist can be administered in combination with other end-stage liver disease therapeutics. End-stage liver disease therapeutics include, but are not limited to Pegylated Interferon (Pegasys), Interferon alpha, loop diuretics, potassium-sparing diuretics, hepatitis B Immune Globulin, gi cathartics, milk thistle, sodium benzoate, zinc, rifiximine, rifampin, terlipressin, ornipressin, glucocorticoids, beta blockers, opioid antagonists, barbiturates, human albumin solution, non-absorbable oral antibiotics, fluoroquinolones, cephalosporins, bile acids, fluorine, oral and systemic vasoconstrictors, iron and copper chelation therapy.

The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carriers include, but are not limited to, sterile water, saline, Ringer's solution, dextrose solution, and buffered solutions at physiological pH. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the polynucleotide, polypeptide, antibody, T-cell, TCR, or APC compositions disclosed herein. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The antagonists disclosed herein may also be formulated as liposomes. Liposomes containing the antagonist are prepared by methods known in the art, such as described in Epstein et al., Proc. Natl. Acad. Sci. USA, 82:3688 (1985); Hwang et al., Proc. Natl. Acad. Sci. USA, 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556 and are further described elsewhere herein.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst. 81(19)1484 (1989).

### B. METHODS OF INHIBITING ANGIOGENESIS

Disclosed herein are methods of using angiogenesis antagonists, such as the angiogenesis antagonists disclosed herein. In some aspects, the methods comprise inhibiting angiogenesis. In some aspect, the methods comprise inhibiting vasculogenesis. In some aspects, the methods comprise inhibiting angiogenesis of a vein, artery, venule, or capillary network. In some aspects, the methods comprise modulating vascular remodeling or sprouting. In some aspects, the methods comprise modulating endothelial cell hypertrophy, hyperplasia, recruitment, or survival. In some aspects, the methods comprise modulating smooth muscle cell hypertrophy, hyperplasia, recruitment, or survival. In some aspects, the methods comprise modulating vascular permeability.

For example, disclosed herein are methods of using angiogenesis antagonists for treating or preventing diseases associated with abberant angiogenesis. Abberant angiogenesis is known to be involved in many diseases such as macular degeneration and diabetic retinopathy.

1. Angiogenesis Antagonists

The angiogenesis antagonist of the disclosed uses and methods can be any angiogenesis antagonist described herein. The angiogenesis antagonist of the disclosed uses and methods can be any angiogenesis antagonist identified with the ability to inhibit VEGF-mediated vascularization. Thus, the angiogenesis antagonist of the disclosed uses and methods can be any composition identified with the ability to inhibit VEGF activity. For example, the angiogenesis antagonist can be a VEGF antagonist. The VEGF antagonist can be, for example, an antibody that blocks the binding of VEGF to VEGF-R (Flt-1 and/or Flk-1/KDR). For example, the antibody can be an anti-VEGF antibody, such as bevacizumab. The VEGF antagonist can be a dithiocarbamate. The VEGF antagonist can be zinc or a pharmaceutically acceptable salt or chelate thereof (e.g., zinc gluconate, zinc acetate, zinc sulfate, or zinc chloride). The VEGF antagonist can comprise zinc that is separately administered to a subject with a zinc ionophore.

The VEGF antagonist can comprise two or more compositions that are separately administered. Thus, the disclosed methods can comprise separately administering to the subject a first composition and a second composition, wherein the first and second composition accumulate in the liver to act as a VEGF anatagonist.

For example, the first and second compositions can be zinc and a zinc ionophore. The first and second compositions can be zinc and disulfuram. The first and second compositions can be dithiocarbamate and a metal ion. The first and second compositions can be zinc gluconate and disulfuram. The first and second compositions can be zinc acetate and disulfuram. The first and second compositions can be zinc sulfate and disulfuram. The first and second compositions can be zinc chloride and disulfuram.

The first and second compositions can be zinc gluconate and diethyldithiocarbamate. The first and second compositions can be zinc acetate and diethyldithiocarbamate. The first and second compositions can be zinc sulfate and diethyldithiocarbamate. The first and second compositions can be zinc chloride and diethyldithiocarbamate

Thus, wherein “VEGF antagonist” is used herein, also disclosed are two or more compositions that are separately administered and combine in the liver to form a VEGF antagonist. For example, a therapeutically effective does of a VEGF antagonist includes doses of separately administered compositions that combine in the liver at therapeutically effective amounts.

In some aspects, the time interval between administration of the first and second compositions is determined based on the time necessary for the first composition to substantially clear the gastrointestinal system and enter the blood stream. Thus, in some aspects, the first and second compositions are administered at least about 0.5, 1.0, 1.5, 2.0, 2.5, 3.0, 3.5, 4.0, 4.5, 5.0, 5.5, 6.0 hours apart.

In some aspects, the angiogenesis antagonist of the disclosed uses and methods is a dithiocarbamate given in combination with a heavy metal ion, such as zinc.

In some aspects, the angiogenesis antagonist of the disclosed uses and methods is a copper chelator.

In some aspects, the angiogenesis antagonist of the disclosed uses and methods is zinc given in combination with a zinc ionophore, such as a dithiocarbamate. Non-limiting examples of dithiocarbamates include pyrrolidine dithiocarbamate, diethyldithiocarbamate, disulfuram, and dimethyldithiocarbamate.

Zinc can be administered separately as an aqueous solution. In the case of charged zinc ion coordination complexes, the zinc ions can be administered in a pharmaceutically suitable form. Ideally, the zinc ions are coordinated to a chelating agent such as acetate, lactonate, glycinate, citrate, propionate, or gluconate, with a pharmaceutically acceptable counter ion. In some aspects, the amount of zinc ion to be used is proportional to the amount of dithiocarbamate to be administered based on the stoichiometric ratio between a metal ion and the dithiocarbamate. In some aspects, zinc is administered to the subject first, and disulfuram is administered to the subject after a time sufficient for a substantial amount of the zinc to have passed out of the gastrointestinal system and into the blood stream.

Effective dosages and schedules for administering the compositions may be determined empirically, and making such determinations is within the skill in the art. The dosage ranges for the administration of the compositions are those large enough to produce the desired effect in which the symptoms and disorders are affected. The dosage should not be so large as to cause adverse side effects, such as unwanted cross-reactions, anaphylactic reactions, and the like. Generally, the dosage will vary with the age, condition, sex and extent of the disease in the subject, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Depending on the type and severity of the disease, about 1 μg/kg to 50 mg/kg (e.g. 0.1-20 mg/kg) of antagonist is an initial candidate dosage for administration to the subject, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. Optionally, the antagonist can be administered every two to three weeks, at a dose ranged from about 1.5 mg/kg to about 15 mg/kg. Optionally, such dosing regimen can be used in combination with another end-stage liver disease therapeutic.

As noted above, however, these suggested amounts of antagonist are subject to a great deal of therapeutic discretion. The key factor in selecting an appropriate dose and scheduling is the result obtained, as indicated above. For example, relatively higher doses may be needed initially for the treatment of ongoing and acute diseases. To obtain the most efficacious results, depending on the disease or disorder, the antagonist is administered as close to the first sign, diagnosis, appearance, or occurrence of the disease or disorder as possible or during remissions of the disease or disorder.

Following administration of a disclosed composition, the efficacy of the therapy can be assessed in various ways well known to the skilled practitioner.

The compositions disclosed herein may be administered prophylactically to subjects or subjects who are at risk for diseases or complications associated with aberrant angiogenesis.

Aside from administration of protein antagonists to a subject, the angiogenesis antagonists can be administered by gene therapy. Such administration of nucleic acid encoding the angiogenesis antagonist is encompassed by the expression “administering a therapeutically effective amount of an antagonist”. See, for example, WO96/07321 published Mar. 14, 1996 concerning the use of gene therapy to generate intracellular antibodies. Nucleic acids and methods of administering a nucleic acid encoding the angiogenesis antagonist are described above.

2. Diabetic Retinopathy

Disclosed herein is the use of an angiogenesis antagonist disclosed herein in the preparation of a medicament for the treatment of diabetic retinopathy in a subject. Thus, also disclosed are methods of treating a subject with diabetic retinopathy, comprising administering to the subject an effective amount of an angiogenesis antagonist disclosed herein.

Diabetic retinopathy is retinopathy (damage to the retina) caused by complications of diabetes mellitus, which can eventually lead to blindness. It is an ocular manifestation of systemic disease which affects up to 80% of all patients who have had diabetes for 10 years or more. Despite these intimidating statistics, research indicates that at least 90% of these new cases could be reduced if there was proper and vigilant treatment and monitoring of the eyes.

Diabetic retinopathy is the result of microvascular retinal changes. Hyperglycemia-induced pericyte death and thickening of the basement membrane lead to incompetence of the vascular walls. These damages change the formation of the blood-retinal barrier and also make the retinal blood vessels become more permeable.

Small blood vessels—such as those in the eye—are especially vulnerable to poor blood sugar (blood glucose) control. An overaccumulation of glucose and/or fructose damages the tiny blood vessels in the retina. During the initial stage, called nonproliferative diabetic retinopathy (NPDR), most people do not notice any change in their vision.

Some people develop a condition called macular edema. It occurs when the damaged blood vessels leak fluid and lipids onto the macula, the part of the retina that lets us see detail. The fluid makes the macula swell, which blurs vision.

As the disease progresses, severe nonproliferative diabetic retinopathy enters an advanced, or proliferative, stage. The lack of oxygen in the retina causes fragile, new, blood vessels to grow along the retina and in the clear, gel-like vitreous humour that fills the inside of the eye. Without timely treatment, these new blood vessels can bleed, cloud vision, and destroy the retina. Fibrovascular proliferation can also cause tractional retinal detachment. The new blood vessels can also grow into the angle of the anterior chamber of the eye and cause neovascular glaucoma. Nonproliferative diabetic retinopathy shows up as cotton wool spots, or microvascular abnormalities or as superficial retinal hemorrhages. Even so, the advanced proliferative diabetic retinopathy (PDR) can remain asymptomatic for a very long time, and so should be monitored closely with regular checkups.

Thus, disclosed herein is a method of treating diabetic retinopathy in a subject comprising administering to the subject an effective amount of a pharmaceutical composition comprising an angiogenesis antagonist and a pharmaceutically acceptable carrier to the subject. For example, disclosed herein is a method of treating diabetic retinopathy in a subject comprising administering to the subject an effective amount of a pharmaceutical composition comprising dithiocarbamate and a heavy metal ion. In some aspects the dithiocarbamate is disulfuram. For example, disulfuram can be separately administered with a heavy metal ion to a subject at currently approved doses for alcoholism. The metal ion can be coordinated to a chelating agent such as acetate, lactonate, glycinate, citrate, propionate, or gluconate, with a pharmaceutically acceptable counter ion. In some aspects, the heavy metal ion is zinc. Thus, in some aspects, disulfuram is separately administered with chelated zinc (e.g., zinc gluconate, zinc acetate, zinc sulfate, or zinc chloride) to the subject. Thus, disclosed herein is a method of treating diabetic retinopathy in a subject, comprising administering to the subject an effective amount of a heavy metal ion, such as zinc, and an effective amount of a pharmaceutical composition comprising a dithiocarbamate, such as disulfuram. For example, the heavy metal ion can be administered orally while the dithiocarbamate is administered to the eye of the subject. Preferably, if the heavy metal and dithiocarbamate are both orally administered, they are separately administered.

3. Macular Degeneration

Thus, disclosed herein is the use of an angiogenesis antagonist in the preparation of a medicament for the treatment of macular degeneration in a subject. Thus, also disclosed are methods of treating a subject with macular degeneration, comprising administering to the subject an effective amount of an angiogenesis antagonist.

Age-related macular degeneration (AMD) is the most frequent cause of legal blindness in the elderly in industrial countries (Van Leeuwen et al. (2003) European Journal of Epidemiology 18: 845-854). It is a heterogeneous disease, which is characterized by progressive loss of central, high acuity vision. For the patient it compromises dramatically quality of life, since they lose their ability to read, to recognize faces and day-to-day tasks become major obstacles. According to the WHO a total of 30-50 million individuals are affected and about 14 million people are blind or severely visually impaired due to AMD (Gehrs et al., (2006) Annals of Medicine 38:450-471).

The most prominent clinical and histopathological lesions of AMD involve the choriocapillaris, Bruch's membrane, and the retinal pigment epithelium (RPE) (Ambati et al. (2003) Survey of Opthalmology 48:257-293). The choriocapillaris is a highly specialized capillary plexus that interacts with the highly metabolic active RPE. The RPE forms the outer blood-retina barrier and supplies the photoreceptors, the sensory cells in the eye, with nutriments as well as phagocytes daily shed outer photoreceptor segments which are degraded and partially recycled. Under normal conditions unrecycled end products are rendered into the choriocapillaris. Bruch's membrane is a five layer connective tissue between the RPE and choriocapillaris resembling an arterial intima in its function (Curcio et al. (2001) Invest Opthalmol Vis Sci 42:265-274). With age Bruch's membrane undergoes distinctive degenerative changes. One major characteristic feature next to thickening is the accumulation of neutral lipids, which build up a diffusion barrier between the RPE and choriocapillaris compromising RPE and photoreceptor function (Curcio et al. (2001) Invest Opthalmol Vis Sci 42:265-274; Pauleikhoff et al. (1990) Opthalmology 97:171-178; Moore et al. (1995) Invest Opthalmol Vis Sci 36:1290-1297).

In early stages of AMD an additional deposition of debris is observed between the basal membrane of the RPE (1^{st} layer of Bruch's membrane) and the inner collagenous layer (2^{nd} layer of Bruch's membrane). This debris is called basal linear deposits and drusen, both rich in lipids and hallmarks of AMD, impairing even more the diffusion along Bruch's membrane (Gehrs et al, (2006) Annals of Medicine 38:450-471; Curcio et al. (1999) Arch Opthalmol 117:329-339; Curcio et al. (2005) Experimental Eye Research 81: 731-741; Haimovici et al. (2001) Invest Opthalmol Vis Sci 42:1592-1599). Furthermore, cytotoxic and lipid rich, metabolic end products, called lipofuscin, accumulate in the RPE cells (Beatty et al. (2000) Sury Opthalmol 45:115-134). All these conditions together cause oxidative stress and inflammation resulting in RPE atrophy and successively photoreceptor degeneration (Kopitz et al. (2004) Biochimie 86: 825-831). This atrophy of RPE and photoreceptors is called the dry form of AMD and progresses slowly and irreversibly. Currently a treatment or prevention of this form of AMD, which affect about 85-90% of all AMD patients, does not exist (Van Leeuwen et al. (2003) European Journal of Epidemiology 18: 845-854).

The second form of AMD is called wet AMD and can arise from the dry form. It affects about 10-15% of all AMD patients and is marked by the growth of a pathological vessel from the choriocapillaris into the subretinal space, called choroidal neovascularization (CNV) (Gehrs et al. (2006) Annals of Medicine 38:450-471 and Ambati et al. (2003) Survey of Opthalmology 48:257-293). It causes a rapid, irreversible vision loss due to leakage, bleeding, and scaring (Ambati et al. (2003) Survey of Opthalmology 48:257-293).

Antiangiogentic therapies have been developed targeting vascular endothelial growth factor, which can show success in slowing down the progression of vision loss (Michels et al. (2006) Expert Opin Investig Drugs 15:779-793). In general, current therapies use antibodies or antibody fragments against VEGF, which are injected into the vitreous body of the eye (Michels et al. (2006)). A prevention therapy of wet AMD does not exist (Gehrs et al. (2006)), which would be especially desirable when the vision in one eye is already largely compromised and the second eye shows definite risk factors for a progression like e.g. large soft drusen (Ambati et al. (2003)).

Neovascular or exudative AMD, the wet form of advanced AMD, causes vision loss due to abnormal blood vessel growth in the choriocapillaries, through Bruch's membrane, ultimately leading to blood and protein leakage below the macula. Bleeding, leaking, and scarring from these blood vessels eventually cause irreversible damage to the photoreceptors and rapid vision loss if left untreated.

Until recently, no effective treatments were known for wet macular degeneration. However, new drugs, called anti-angiogenics or anti-VEGF (anti-Vascular Endothelial Growth Factor) agents, when injected directly into the vitreous humor of the eye using a small, painless needle, can cause regression of the abnormal blood vessels and improvement of vision. The injections frequently have to be repeated on a monthly or bi-monthly basis. Examples of these agents include Lucentis, Avastin and Macugen. Only Lucentis and Macugen are FDA approved as of April 2007. Macugen has been found to have only minimal benefits in neovascular AMD and is no longer used. Worldwide,
Avastin has been used extensively despite its “off label” status. The cost of Lucentis is approximately $2000 US while the cost of Avastin is approximately $150.

The remodeling of Bruch's membrane provides an undisturbed passage between retinal pigment epithelium and choriocapillaris, which is essential for the health of the retina. The retinal pigment epithelium stands with the choriocapillaris in a close relationship and they are dependent on each other. An uncompromised communication between these structures improves the blood supply for the outer retina by the choriocapillaris and the retinal pigment epithelium layer integrity by improved anchorage on Bruch's membrane via water soluble proteins.

The same mechanism applies to wet AMD as for dry AMD. Due to the improved environmental conditions retinal pigment epithelium cells also reduce the secretion of pro-angiogenic factors, which normally keeps a neovascularization active for a longer period. In combination with anti-angiogenic treatments (elsewhere herein) pro-angiogenic mechanisms are not just temporarily blocked but the secretion stimulus can be long-term reduced.

Thus, in certain embodiments, this invention contemplates administering one or more of the active agents described herein to a subject at risk for, or incurring, one or more of the symptoms and/or at risk for or incurring a symptom of an eye disease and/or an associated pathology (e.g., blindness).

Thus, for example, a person having or at risk for eye disease may prophylactically be administered a one or more active agents of this invention. A person (or animal) subject to an eye disease, e.g., macular degeneration, can be treated with a one or more agents described herein to mitigate or prevent the development of eye disease. A person (or animal) subject to trauma, e.g., acute injury, tissue transplant, etc. can also be treated with a polypeptide of this invention to mitigate the development of eye disease.

In certain instances such methods will entail a diagnosis of the occurrence or risk of an eye disease. The eye disease typically involves alterations in drusen, basal linear deposit, basal laminar deposit, lipid accumulation in and/or Bruch's membrane.

Another theory could be that with macular degeneration, where the presence of lipids in the Bruch's membrane causes the transfer of blood from the eye vessels through the Bruch's membrane to the retinal pigment cells and then to the photoreceptors to decrease. The decrease in blood flow leads to a decrease in oxygen getting to the photoreceptors. The body then responds by creating more vasculature that invades the Bruch's membrane and into the retinal pigment epithelial cells to compensate for the decrease in oxygen supply to the photoreceptors and retinal pigment epithelial cells. By providing one or more of the active agents described herein, the lipid accumulation could be removed and/or prevented, thereby relieving the need for increased vasculature. In addition, by providing one or more of the active agents described herein in combination with an anti-angiogenic factor, not only could the lipid accumulation be removed and/or prevented, the revascularization could be prevented as well, thereby relieving the need for increased vasculature and preventing detrimental vascular growth.

Thus, disclosed herein is a method of treating AMD in a subject comprising administering to the subject an effective amount of a pharmaceutical composition comprising an angiogenesis antagonist and a pharmaceutically acceptable carrier to the subject. For example, disclosed herein is a method of treating AMD in a subject comprising administering to the subject an effective amount of a pharmaceutical composition comprising dithiocarbamate and a heavy metal ion. In some aspects the dithiocarbamate is disulfuram. For example, disulfuram can be separately administered with a heavy metal ion to a subject at currently approved doses for alcoholism. The metal ion can be coordinated to a chelating agent such as acetate, lactonate, glycinate, citrate, propionate, or gluconate, with a pharmaceutically acceptable counter ion. In some aspects, the heavy metal ion is zinc. Thus, in some aspects, disulfuram is separately administered with chelated zinc (e.g., zinc gluconate, zinc acetate, zinc sulfate, or zinc chloride) to the subject. Thus, disclosed herein is a method of treating AMD in a subject, comprising administering to the subject an effective amount of a heavy metal ion, such as zinc, and an effective amount of a pharmaceutical composition comprising a dithiocarbamate, such as disulfuram. For example, the heavy metal ion can be administered orally while the dithiocarbamate is administered to the eye of the subject. Preferably, if the heavy metal and dithiocarbamate are both orally administered, they are separately administered.

4. Liver Disease

As disclosed herein, abberant angiogenesis is also involved in liver disease. In some aspects, the liver disease is chronic inflammatory liver disease, fatty liver disease, or end-state liver disease. In some aspects, the liver disease is a non-alcohol liver disease. In some aspects, the liver disease is chronic active liver disease from hepatitis B, C D or E. In some aspects, the liver disease is cryptogenic hepatitis with cirrhosis. In some aspects, the liver disease is primar biliaruy cirrhosis. In some aspects, the liver disease is automimmune hepatitis. In some aspects, the liver disease is sclerosing cholangitis. In some aspects, the liver disease is graft ver host liver disease. In some aspects, the liver disease is alpha-1-antitrypsin deficiency-associated liver disease. In some aspects, the liver disease is hemochromatosis (iron overload). In some aspects, the liver disease is Wilson's disease (copper overload). In some aspects, the liver disease is alcoholic cirrhosis. In some aspects, the liver disease is nonalcoholic fatty liver or steatosis. In some aspects, the liver disease is from sarcoidosis. In some aspects, the liver disease is from amyloidosis. In some aspects, the liver disease is portal hypertension from portal vein thrombosis.

For example, disclosed herein is the use of an angiogenesis antagonist in the preparation of a medicament for the treatment of end-stage liver disease in a subject. Thus, also disclosed are methods of treating a subject with end-stage liver disease, comprising administering to the subject an effective amount of an angiogenesis antagonist.

Also disclosed herein is the use of an angiogenesis antagonist in the preparation of a medicament for the treatment of inflammatory liver disease. Thus, also disclosed are methods of treating a subject with inflammatory liver disease, comprising administering to the subject an effective amount of an angiogenesis antagonist.

Also disclosed are methods of treating a subject with end-stage liver disease comprising administering to the subject an effective amount of a pharmaceutical composition comprising an angiogenesis antagonist and a pharmaceutically acceptable carrier to the subject.

Also disclosed are methods of reducing, preventing, and/or treating end-stage liver disease complications in a subject. For example, disclosed are methods of reducing, preventing, and/or treating end-stage liver disease complications in a subject comprising administering to the subject an effective amount of an angiogenesis antagonist to the subject. Also disclosed are methods of reducing, preventing, and/or treating end-stage liver disease complications in a subject comprising administering to the subject an effective amount of a pharmaceutical composition comprising an angiogenesis antagonist and a pharmaceutically acceptable carrier to the subject.

The various end-stage liver disease complications to be treated, prevented, or reduced herein are described above. Examples of end-stage liver disease complications include, but are not limited to portal-systemic collaterals and gastrointestinal hemorrhages

This methods disclosed herein can also be used to abrogate the formation of additional portal-systemic collaterals in the setting of end-stage liver disease through the use of an angiogenesis antagonist. As a consequence of altered flow dynamics within the diseased liver, formation of new portal-systemic collaterals contributes to the increased venous capacitance seen in end-stage liver disease and can incite the inevitable cycle of hemodynamic derangement observed in end-stage liver disease. As such, methods disclosed herein can also be used to ameliorate consequences of increased venous capacitance such as hyperdynamic circulation, hypermetabolism, muscle wasting, ascites, edema, renal failure, hepatic hydrothorax, heptopulmonary syndrome, portopulmonary hypertension, as wells as variceal and gastrointestinal hemorrhage. For example, disclosed are methods of preventing the formation of portal-systemic collaterals in a subject comprising administering to the subject an effective amount of an angiogenesis antagonist.

The subject of the herein disclosed uses and methods can be a mammal, especially humans, with complications of end-stage liver disease. The subject can be identified by a combination of a medical history and physical exam. Pertinent historical items of interest include a history of viral hepatitis exposure, prolonged alcohol use, environmental toxin exposure, parasitic infection, a congenital condition resulting in end-stage liver disease (alpha-1 anti-trypsin disease, Wilson's disease, hemochromatosis, non-alcoholic fatty liver disease, metabolic storage diseases, primary sclerosing choangitis, neonatal hepatitis, biliary atresia, choledocal cyst, Byler's disease, cholestatic diseases of infancy, and mitochondrial disorders. Symptoms include abnormal bleeding and bruising, hemetemesis, melena, hematochezia, jaundice, fatigue, muscle wasting, sleep-wake disturbances, malnutrition, ascites, peripheral edema, pulmonary edema, pruritis, encephalopathy. Corroborating physical signs include jaundice, scleral icterus, gynecomastia, hemorrhoids, ascites, splenomegaly, peripheral edema, muscle wasting, palmar erythema, Depuytren's contracture, abdominal bruits, spider telengiectasias, altered mental status, asterixis, testicular atrophy, cold extremities, tachycardia, and hypotension. Confirmation can be made by liver biopsy demonstrating end-stage liver disease or imaging (CT, MRI, ultrasound) demonstrating portal-systemic varices, splenomegaly, and an atrophied liver with irregular contour.

Additional radiographic studies include of endoscopic retrograde cholangiopancreatography (ERCP) as well and percutaneous transhepatic cholangiography (PTC). Laboratory evidence of end-stage liver disease includes components of the Model for End-Stage Liver Disease (MELD) scoring system which include total bilirubin, creatinine, and international normalized ratio (INR). Other biological markers include, but are not limited to platelet count, serological testing for hepatitis A-E; parasitic testing; iron and copper studies; chromosomal studies; quantitative assessment of components of the tricarboxylic acid, Cori, and urea cycles as well as the electron transport chain; alpha-1-antitrypsin levels; serum anti-nuclear antigen/antibody; anti-mitochondirial antigen/antibody; and liver-kidney microsomal antibody. Identification of candidates can also be achieved through the measurement of the hepatic vein wedge pressure gradient as well as the documentation of gastrointestinal varices using upper and lower endoscopy.

For the prevention, treatment, or reduction of end-stage liver disease or end-stage liver disease complications, the appropriate dosage of antagonist will depend on the type of disease to be treated, as defined above, the severity and course of the disease, whether the antagonist is administered for preventive or therapeutic purposes, previous therapy, the patient's clinical history and response to the antagonist, and the discretion of the attending physician. The antagonist is suitably administered to the patient at one time or over a series of treatments.

In a combination therapy regimen, the compositions of the present invention are administered in a therapeutically effective or synergistic amount. As used herein, a therapeutically effective amount is such that co-administration of the antagonist and one or more other therapeutic agents, or administration of a composition of the present invention, results in reduction or inhibition of the targeting disease or condition. A therapeutically synergistic amount is that amount of antagonist and one or more other therapeutic agents necessary to synergistically or significantly reduce or eliminate conditions or symptoms associated with a particular disease.

### C. METHODS OF INHIBITING LIVER INFLAMMATION

Currently, anti-inflammatory agents, with few exceptions, have generally not been effective in cases of acute and chronic liver inflammation. These exceptions include the use of steroids and immunosuppressives in the setting of autoimmune conditions like primary biliary cirrhosis as well as interferons and ribavirin in the setting of hepatitis C. In the latter condition, results are mostly attributed to interferon's anti-viral properties, rather than a direct anti-inflammatory effect. Moreover, in some cases, interferon use has been known to accelerate inflammation as well hepatic decompensation.

The final common pathway leading to cirrhosis and eventually hepatic transplantation finds its origin in the multitude of acute and chronic inflammatory conditions affecting the liver. For instance, diseases with acute and chronic necroinflammatory components account for approximately 95% of known liver disease. For years, chronic active hepatitis (CAH), a manifestation of many of the viral hepatitides, accounted for the majority of these cases. At the present time, acute and chronic viral hepatitis accounts for approximately 30% of liver transplants performed in this country. It is now estimated that 10-20% of Americans now harbor fatty infiltration of their liver and 2-5% actually have evidence of associated inflammation. It is currently the 2nd leading cause for liver transplantation in the United States and is only expected to increase. However, even as non-alcoholic steatohepatitis (NASH) is becoming increasingly more prevalent in the United States, viral hepatitides continue to wreck havoc worldwide.

As a liver disease, NASH, resembles alcoholic liver disease but occurs in patients who drink little or no alcohol. NASH occurs most often in adults over the age of 40 who are overweight or have diabetes, insulin resistance (pre-diabetes), or hyperlipidemia (excess concentrations of fatty materials in the blood). NASH can also occur in children, the elderly, normal-weight, and non-diabetic persons. Almost all patients with NASH are insulin resistant to some degree. However, only a minority of patients who are insulin resistance develop NASH. While an increased amount of fat in the liver may in itself lead to inflammation, no evidence suggests that insulin resistance alone can lead to NASH.

The process whereby liver inflammation and death of liver tissue develop in NASH remains to be clearly explained. Several theories, however, have been advanced. First, it is possible that the accumulation of fat in the liver alone could lead to the development of NASH. According to this theory, the large quantity of fat in the liver is thought to be a source of peroxidation (removal of electrons from molecules). Peroxidation thereby generates so-called free radicals. These free radicals then damage proteins and organelles (small structures within a cell) in the liver cells. Finally, this damage leads to cell death and/or an inflammatory cell cascade that removes the afflicted cells. In other words, the fat could be thought of as potential fuel waiting to be ignited.

However, a growing body of work in animal models of fatty liver suggests a two-hit hypothesis. With this theory, the first hit is the fatty liver (steatosis). Then, a second event, or second hit, leads to the development of NASH. Multiple potential second hits have been suggested, including:

a) small hormones (cytokines), such as tumor necrosis factor-alpha, which is secreted by cells and involved in inflammation, may induce cell death and even increase insulin resistance;

b) intracellular organelles (mitochondria) that provide energy to the cell may malfunction and thereby cause a decrease in cell energy and lead to cell death;

c) enzymes (cytochromes) that are involved in multiple metabolic pathways may lead to increased peroxidation and its consequences, as described above; and

d) receptors in the cell nucleus that are involved in triggering the effects of insulin (peroxisome proliferator activating receptors, PPAR) may fail and thus lead to insulin resistance, inflammation of the liver, and scarring of the liver.

The development of severe, irreversible scarring of the liver (cirrhosis) in NASH is even more poorly understood than the development of liver inflammation and death of liver tissue, as discussed above. Cirrhosis may simply develop over time as a result of chronic inflammation and repair, or may be due to yet, a third hit.

As disclosed herein, diseases with an inimitable propensity to induce acute and chronic necroinflammatory activity in the liver are potentially impacted by strong anti-inflammatory agents with a unique predilection for hepatic uptake and concentration.

Thus, as disclosed herein, dithiocarbamates such as disulfuram (DSF, tetraethylthiuram disulfide), a carbamate derivative with exceptional anti-inflammatory properties and exclusive hepatic metabolism, is a useful adjunct in the clinical setting to ameliorate many of the conditions that may ultimately progress to cirrhosis. Thus, provided is a method of treating or preventing liver disease in a subject, comprising administering to the subject a therapeutically effective amount of a dithiocarbamate.

1. Dithiocarbamate

Disulfuram (Antabuse™) has been approved for clinical use by the FDA since 1948 and has been used primarily by the addiction community since that time for its alcohol-adversative properties. By exploiting its innate ability to interfere with aldhehyde dehydrogenase, disulfuram is given to alcoholics who are then subjected to a severe reaction characterized by flushing, a racing heartbeat, and a drop in blood pressure that causes dizziness. Other unpleasant symptoms include headache, shortness of breath, palpitations, nausea and vomiting after even the slightest exposure to ethanol. Its use has fallen out of favor in recent years and it is often circumvented by those in whom it is intended to treat simply by deciding not to take the medication.

As disclosed herein, disulfuram, with its unique pharmacokinetic properties and ability to interfere with NF-KB translocation and DNA binding can have significant anti-inflammatory properties concentrated within the hepatic parenchyma. Disulfuram also inhibits several caspases important for inflammation, among them caspase-1. As disclosed herein, these effects are even more pronounced in the setting of a divalent metal, like zinc (zinc gluconate, zinc acetate, zinc sulfate, or zinc chloride). Resultingly, diseases manifesting themselves uniquely in the form of hepatic inflammation such as NASH and chronic active hepatitis should be subject to inhibition by its administration. Others such as fulminant hepatic failure, Reye's syndrome, and acute allograft rejection are additional entities that should respond. As there are few effective therapies for treatment of acute hepatitis of any kind, the ability to mitigate acute and chronic necroinflammatory activity in the liver represent the first effective treatment addressing the pathological changes that eventually lead to cirrhosis or potentially, fulminant hepatic failure.

Dithiocarbamates are a broad class of molecules that have the ability to chelate metal ions, as well as react with sulfhydryl groups and glutathione. After metal-mediated conversion to their corresponding disulfides, dithiocarbamates inhibit cysteine proteases by forming mixed disulfides with critical protein thiols.

In addition to their reduced thioacid form, dithiocarbamates can also or are known to exist in four other forms: a) the disulfide, a condensed dimer of the thioacid with elimination of reduced sulfhydryl groups by disulfide bond formation; b) the negatively charged thiolate anion, generally as a salt, such as the sodium salt or ammonium salt; c) the 1,1-dithiolato coordination complex of metal ions in which the two adjoining sulfur atoms of the dithiocarbamate are bound to the same metal ion, for example, titanium(III), vanadium(III), chromium(III), iron(III), cobalt(III), nickel(II), copper(II), silver(I), gold(III), Zn(II), Au(I), Mn(III), Ga(III), Pt(II); and d) the monodentate dithiolato coordination complex in which either one of the sulfur atoms binds to a metal ion, for example titanium(III), vanadium(III), chromium(III), iron(III), cobalt(III), nickel(II), copper(II), silver(I), or gold(III). The disulfide, thiolate anion, and coordination complexes of dithiocarbamates are all structurally distinct from the reduced form of PDTC used by Chinery, et al., in that they have no reduced sulfhydryl molecular moiety and are incapable of functioning as antioxidants by donating the proton from a reduced sulfhydryl to scavenge electrons of free radical species.

Thus, in some aspects, the angiogenesis inhibitor of the disclosed compositions and methods is a dithiocarbamate disulfide. Thus, in some aspects, the angiogenesis inhibitor of the disclosed compositions and methods is a thiolate anion. Thus, in some aspects, the angiogenesis inhibitor of the disclosed compositions and methods is a coordination complex.

In some aspects, the dithiocarbamates of the disclosed compositions and methods can be identified and/or selected based on its ability to block nuclear factor-KB (NF-KB).

In some aspects, the angiogenesis antagonist of the disclosed compositions and methods is a dithiocarbamate thiolate anion. As is known in the art, dithiocarbamates react with critical thiols and also complex metal ions. Thus, the dithiocarbamate of the disclosed compositions and methods can be a coordination compound.

However, dithiocarbamates and metal ions can have deleterious effects when co-administrated. Thus, in other aspects, the dithiocarbamate of the disclosed compositions and methods is separately administered to a subject with a metal ion. In some aspects, the separately administered dithiocarbamates and metal ions accumulate in the liver and there form a coordination compound.

A therapeutically effect amount of the herein disclosed dithiocarbamate anion compound and an intracellular metal ion stimulant, which can enhance the intracellular level of the above described metal ions in the liver of the subject, can therefore be separately administered to a subject. Intracellular heavy metal ion carriers are known. For example, ceruloplasmin can be administered to the patient to enhance the intracellular copper level. Other metal ion carriers known in the art may also be administered in accordance with this aspect of the invention. The heavy metal ion carriers and the dithiocarbamate disulfide or metal anion can be administered together or separately.

Ceruloplasmin is a protein naturally produced by the human body and can be purified from human serum. This 132-kD glycoprotein, which carries 7 copper(II) ions complexed over three 43-45 kD domains, is an acute phase reactant and the major copper-carrying protein in human plasma. See Halliwell, et al., Methods Enzymol. 186:1-85 (1990). When transported into cells, at least some of the bound copper(II) ions can be accessible for complexation with the dithiocarbamate disulfide or thiolate anion administered to the patient. (See Percival, et al., Am. J. Physiol. 258:3140-3146 (1990).) Ceruloplasmin and dithiocarbamate disulfides or thiolate anions are typically administered in different compositions. Dithiocarbamate disulfides or thiolate anions can be administered at about the same time, or at some time apart. For example, ceruloplasmin can be administered from about five minutes to about 12 hours before or after dithiocarbamate disulfide or thiolate anions are administered to the patient.

In some aspects the dithiocarbamate is disulfuram. For example, disulfuram can be separately administered with a heavy metal ion to a subject at currently approved doses for alcoholism. The metal ion can be coordinated to a chelating agent such as acetate, lactonate, glycinate, citrate, propionate, or gluconate, with a pharmaceutically acceptable counter ion. In some aspects, the heavy metal ion is zinc. Thus, in some aspects, disulfuram is separately administered with chelated zinc (e.g., zinc gluconate, zinc acetate, zinc sulfate, or zinc chloride) to the subject.

In some aspects, zinc is administered first, and disulfuram is administered after a time sufficient for a substantial amount of the zinc to have passed out of the gastrointestinal system into the blood stream.

Disulfuram and its diethyldithiocarbamate anion are effective when administered at amounts within the conventional clinical ranges determined in the art. Disulfuram approved by the U.S. Food and Drug administration (Antabuse™) can be purchased in 250 and 500 mg tablets for oral administration from Odyssey Pharmaceuticals, East Hanover, N.J. 07936. Typically, it is effective at an amount of from about 125 to about 1000 mg per day, preferably from 250 to about 500 mg per day for disulfuram and 100 to 500 mg per day or 5 mg/kg intravenously or 10 mg/kg orally once a week for diethyldithiocarbamate. However, the dosage can vary with the body weight of the patient treated. The active ingredient may be administered at once, or may be divided into a number of smaller doses to be administered at predetermined intervals of time. The suitable dosage unit for each administration of disulfuram is, e.g., from about 50 to about 1000 mg/day, preferably from about 250 to about 500 mg/day. The desirable peak concentration of disulfuram generally is about 0.05 to about 10 μM, preferably about 0.5 to about 5 μM, in order to achieve a detectable therapeutic effect. Similar concentration ranges are desirable for dithiocarbamate thiolate anions and for dithiocarbamate-metal ion chelate compounds.

Disulfuram implanted subcutaneously for sustained release has also been shown to be effective for alcoholism at an amount of 800 to 1600 mg to achieve a suitable plasma concentration. This can be accomplished by using aseptic techniques to surgically implant disulfuram into the subcutaneous space of the anterior abdominal wall. (See e.g., Wilson, et al., J. Clin. Psych. 45:242-247 (1984).) In addition, sustained release dosage formulations, such as an 80% poly(glycolic-co-L-lactic acid) and 20% disulfuram, can be used. The therapeutically effective amount for other dithiocarbamate disulfide compounds can also be estimated or calculated based on the above dosage ranges of disulfuram and the molecular weights of disulfuram and the other dithiocarbamate disulfide compound, or by other methods known in the art.

Minimal side effects on this dosage regimen include a metallic taste in the mouth, flatulence, and intolerance to alcoholic beverages. An enteric-coated oral dosage form of diethyldithiocarbamate anions to liberate active drug only in the alkaline environment of the intestine is preferred because of the potential for liberation of carbon disulfide upon exposure of diethyldithiocarbamate to hydrochloric acid in the stomach. An oral enteric-coated form of sodium diethyldithiocarbamate is available in 125 mg tablets as Imuthiol® through Institute Merieux, Lyon, France.

Metal ions can be administered separately as aqueous solutions. In the case of charged metal ion coordination complexes, the metal ions can be administered in a pharmaceutically suitable form. Ideally, the charged metal species contains the metal ion coordinated to a chelating agent such as acetate, lactonate, glycinate, citrate, propionate, or gluconate, with a pharmaceutically acceptable counter ion. In some aspects, the amount of metal ion to be used is proportional to the amount of dithiocarbamate to be administered based on the stoichiometric ratio between a metal ion and the dithiocarbamate.

2. Liver Disease

In some aspects, the liver disease is chronic inflammatory liver disease, fatty liver disease, or end-state liver disease. In some aspects, the liver disease is a non-alcohol liver disease. In some aspects, the liver disease is chronic active liver disease from hepatitis B, C D or E. In some aspects, the liver disease is cryptogenic hepatitis with cirrhosis. In some aspects, the liver disease is primar biliaruy cirrhosis. In some aspects, the liver disease is automimmune hepatitis. In some aspects, the liver disease is sclerosing cholangitis. In some aspects, the liver disease is graft ver host liver disease. In some aspects, the liver disease is alpha-1-antitrypsin deficiency-associated liver disease. In some aspects, the liver disease is hemochromatosis (iron overload). In some aspects, the liver disease is Wilson's disease (copper overload). In some aspects, the liver disease is alcoholic cirrhosis. In some aspects, the liver disease is nonalcoholic fatty liver or steatosis. In some aspects, the liver disease is from sarcoidosis. In some aspects, the liver disease is from amyloidosis. In some aspects, the liver disease is portal hypertension from portal vein thrombosis.

For example, disclosed herein is the use of a dithiocarbamate in the preparation of a medicament for the treatment of end-stage liver disease in a subject. Thus, also disclosed are methods of treating a subject with end-stage liver disease, comprising administering to the subject an effective amount of an angiogenesis antagonist.

Also disclosed herein is the use of an angiogenesis antagonist in the preparation of a medicament for the treatment of inflammatory liver disease. Thus, also disclosed are methods of treating a subject with inflammatory liver disease, comprising administering to the subject an effective amount of a dithiocarbamate.

Also disclosed are methods of treating a subject with end-stage liver disease comprising administering to the subject an effective amount of a pharmaceutical composition comprising a dithiocarbamate and a pharmaceutically acceptable carrier to the subject.

Also disclosed are methods of reducing, preventing, and/or treating end-stage liver disease complications in a subject. For example, disclosed are methods of reducing, preventing, and/or treating end-stage liver disease complications in a subject comprising administering to the subject an effective amount of a dithiocarbamate to the subject. Also disclosed are methods of reducing, preventing, and/or treating end-stage liver disease complications in a subject comprising administering to the subject an effective amount of a pharmaceutical composition comprising a dithiocarbamate and a pharmaceutically acceptable carrier to the subject.

The subject of the herein disclosed uses and methods can be a mammal, especially humans, with complications of end-stage liver disease. The subject can be identified by a combination of a medical history and physical exam. Pertinent historical items of interest include a history of viral hepatitis exposure, prolonged alcohol use, environmental toxin exposure, parasitic infection, a congenital condition resulting in end-stage liver disease (alpha-1 anti-trypsin disease, Wilson's disease, hemochromatosis, non-alcoholic fatty liver disease, metabolic storage diseases, primary sclerosing choangitis, neonatal hepatitis, biliary atresia, choledocal cyst, Byler's disease, cholestatic diseases of infancy, and mitochondrial disorders. Symptoms include abnormal bleeding and bruising, hemetemesis, melena, hematochezia, jaundice, fatigue, muscle wasting, sleep-wake disturbances, malnutrition, ascites, peripheral edema, pulmonary edema, pruritis, encephalopathy. Corroborating physical signs include jaundice, scleral icterus, gynecomastia, hemorrhoids, ascites, splenomegaly, peripheral edema, muscle wasting, palmar erythema, Depuytren's contracture, abdominal bruits, spider telengiectasias, altered mental status, asterixis, testicular atrophy, cold extremities, tachycardia, and hypotension. Confirmation can be made by liver biopsy demonstrating end-stage liver disease or imaging (CT, MRI, ultrasound) demonstrating portal-systemic varices, splenomegaly, and an atrophied liver with irregular contour.

Additional radiographic studies include of endoscopic retrograde cholangiopancreatography (ERCP) as well and percutaneous transhepatic cholangiography (PTC). Laboratory evidence of end-stage liver disease includes components of the Model for End-Stage Liver Disease (MELD) scoring system which include total bilirubin, creatinine, and international normalized ratio (INR). Other biological markers include, but are not limited to platelet count, serological testing for hepatitis A-E; parasitic testing; iron and copper studies; chromosomal studies; quantitative assessment of components of the tricarboxylic acid, Cori, and urea cycles as well as the electron transport chain; alpha-1-antitrypsin levels; serum anti-nuclear antigen/antibody; anti-mitochondirial antigen/antibody; and liver-kidney microsomal antibody. Identification of candidates can also be achieved through the measurement of the hepatic vein wedge pressure gradient as well as the documentation of gastrointestinal varices using upper and lower endoscopy.

For the prevention, treatment, or reduction of end-stage liver disease or end-stage liver disease complications, the appropriate dosage of a dithiocarbamate will depends on the type of disease to be treated, as defined above, the severity and course of the disease, whether the dithiocarbamate is administered for preventive or therapeutic purposes, previous therapy, the patient's clinical history and response to the antagonist, and the discretion of the attending physician. The dithiocarbamate is suitably administered to the patient at one time or over a series of treatments.

In a combination therapy regimen, the compositions of the present invention are administered in a therapeutically effective or synergistic amount. As used herein, a therapeutically effective amount is such that co-administration of the antagonist and one or more other therapeutic agents, or administration of a composition of the present invention, results in reduction or inhibition of the targeting disease or condition. A therapeutically synergistic amount is that amount of antagonist and one or more other therapeutic agents necessary to synergistically or significantly reduce or eliminate conditions or symptoms associated with a particular disease.

### D. ADMINISTRATION

The angiogenesis antagonist can be administered by any suitable means, including oral, intra-ocular (including intraocular delivery device), parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antagonist may suitably be administered by pulse infusion, e.g., with declining doses of the antagonist. Preferably the dosing is given by injections, most preferably intravenous or subcutaneous injections, depending in part on whether the administration is brief or chronic.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the inflammatory disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated herein by reference in its entirety for its teaching of an approach for parenteral administration.

The pharmaceutical composition may be administered in a number of ways depending on whether local or systemic treatment is desired, and on the area to be treated. Administration may be topically (including vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

### E. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Dithiocarbamates

The ordinary artisan knows the synthetic routes towards the coordination compounds of dithiocarbamates. (e.g., D. Coucouvanis, “The chemistry of the dithioacid and 1,1-dithiolate complexes,” Prog. Inorganic Chem. 11:234-371 (1970); D. Coucouvanis, “The chemistry of the dithioacid and 1,1-dithiolate complexes, 1968-1977,” Prog. Inorganic Chem. 26:302-469 (1978); R. P. Burns, et al., “1,1-dithiolato complexes of the transition metals,” Adv. Inorganic Chem. and Radiochem. 23:211-280 (1980); L. I. Victoriano, et al., “The reaction of copper (II) chloride and tetralkylthiuram disulfides,” J. Coord. Chem. 35:27-34 (1995); L. I. Victoriano, et al., “Cuprous dithiocarbamates. Syntheses and reactivity,” J. Coord. Chem. 39:231-239 (1996).) For example, dithiocarbamate coordination compounds of copper(II), gallium (III), bismuth (III) and gold(III) ions can be conveniently synthesized by mixing, in suitable solvents, disulfuram or sodium diethyldithiocarbamate or alkyl ammonium diethyldithiocarbamate with, e.g., CuSO.sub.4, CuCl.sub.2, Bi(NO.sub.3).sub.3, Ga(NO.sub.3).sub.3, HAuCl.sub.4 or HAuBr.sub.4. Other dithiocarbamate chelate compounds are disclosed in, e.g., D. Coucouvanis, “The chemistry of the dithioacid and 1,1-dithiolate complexes,” Prog. Inorganic Chem. 11:234-371 (1970); D. Coucouvanis, “The chemistry of the dithioacid and 1,1-dithiolate complexes, 1968-1977,” Prog. Inorganic Chem. 26:302-469 (1978); R. P. Burns, et al., “1,1-dithiolato complexes of the transition metals,” Adv. Inorganic Chem. and Radiochem. 23:211-280 (1980); L. I. Victoriano, et al., “The reaction of copper (II) chloride and tetralkylhiuram disulfides,” J. Coord. Chem. 35:27-34 (1995); L. I. Victoriano, et al., “Cuprous dithiocarbamates. Syntheses and reactivity,” J. Coord. Chem. 39:231-239 (1996), which are incorporated herein by reference for the teachings of these methods.

2. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

3. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tent-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

### F. KITS

The materials described above as well as other materials can be packaged together in any suitable combination as a kit useful for performing, or aiding in the performance of, the disclosed method. It is useful if the kit components in a given kit are designed and adapted for use together in the disclosed method.

### G. USES

The disclosed compositions can be used in a variety of ways as research tools. For example, the disclosed compositions and methods can also be used as tools to isolate and test new drug candidates for end-stage liver disease or end-stage liver disease complications.

### H. EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### I. DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

As used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. In one aspect, the subject is a mammal such as a primate or a human. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The term “end-stage liver disease complications” refers to medical and physiological complications associated with end-stage liver disease. Examples of end-stage liver disease complications include, but are not limited to, variceal hemorrhage, exsanguinating gastrointestinal hemorrhages, underfilling as seen in accordance with the “peripheral vasodilation theory”, abnormal sodium handling; renal failure, ascites formation, encephalopathy, hepatorenal syndrome, hepatopulmonary syndrome, altered flow, pressure and resistance in portal circulation, sodium handling, bacterial peritonitis, portal hypertensive gastropathy, irreversible chronic liver injury to hepatic parenchyma, excessive fibrosis, increased resistance to flow, hyperdynamic circulation, malnutrition, caput medusa, septicemia, liver failure, coma, hypermetabolism, hepatopulmonary syndrome, portal pulmonary hypertension, cardiomyopathy, encephalopathy, coagulopathy, jaundice, hyperbilirubinemia, pruritis, sleep wake disturbances, fatigue, muscle wasting, and death.

The term “peripheral vasodilation theory” refers to a theory explaining the global pathophysiology of end-stage liver disease. The “peripheral vasodilation theory” suggests that increased venous capacitance, presumably through splanchnic vasodilation, occurs as a consequence of end stage liver disease. The production of local vasodilators such as nitric oxide have been implicated as causative agents in splanchnic vasodilatation, and are widely assumed to be major effectors. However, their involvement is by no means definitive, as attempts to interdict various vasodilatory pathways have failed to reverse or even retard the progress of portal hypertension. Ultimately, the increase in venous capacitance results in increased pooling of blood within the splanchnic vascular bed, thus reducing effective circulating blood volume, and may as readily attributed to the formation of new vessels as a decrease in vasomotor tone of existing ones. In due course, this reduction in circulating blood volume is manifested in decreased renal blood flow, a reduction in glomerular filtration rate (GFR), activation of sodium-retaining systems such as the renin-angiotensin system, and sympathetic nerve activity all leading to the initiation of sodium retention by the kidney.

As the natural history of the disease progresses, splanchinic pooling persists, neurohumoral excitation increases, natural renal vasoprotective mechanisms are overcome, renal vasoconstriction ensues, more renal sodium is retained, and similar to congestive heart failure, total body water expands as the kidney retains sodium in attempt to compensate for fluid lost by the continued splanchnic pooling The consequence is a positive feedback loop, resulting in the manifestations of end-stage liver disease, ascites production, and ultimately, functional renal failure and death.

The term “angiogenic factor” refers to compositions involved in angiogenesis. Examples of angiogenic factors include, but are not limited to Interferon β, Interferon α, Platelet factor 4, Protamine, angiostatic steroids, TNP-470, angiostatin, thalidomide, 2-methyloxyestradiol, endostatin, cleaved antithrombin III, DBF-maf, Caplostatin, Angiopoietins, matrix metalloproteinase (MMP), fibroblast growth factor-2 (FGF2), platelet-derived growth factor (PDGF), Delta-like ligand 4 (DII4), IL-8, and vascular endothelial growth factor (VEGF)

The term “angiogenesis” refers to the growth of new blood vessels from pre-existing vessels. The term “angiogenesis as used herein also refers to sprouting angiogenesis, intussusceptive angiogenesis and therapeutic angiogenesis.

The term “angiogenesis antagonist” and “angiogenic antagonist” are used interchangeably and refer to a composition capable of blocking, inhibiting, abrogating, interfering or reducing pathological angiogenesis associated with a disease or disorder. Many angiogenesis antagonists have been identified and are known in the arts, including those listed by Carmeliet and Jain (2000). Generally, angiogenesis antagonist is a composition targeting a specific angiogenic factor or an angiogenesis pathway. In certain aspects, the angiogenesis antagonist is a protein composition such as an antibody targeting an angiogenic factor. Examples of “angiogenesis antagonists” include, but are not limited to Velcade® (Bortezomib), Thalidomide®, Avastin® (Bevacizumad), Tarceva®(Erlotinib), Macugen®, Endostatin® (Endostar), Nexavar® (Sorafenib), Revlimid®, Sutent® (Sunitinib) and Lucentis®. One of the most recognized angiogenic factors is VEGF, and one of the most potent angiogenesis antagonists is a neutralizing anti-VEGF antibody.

The terms “VEGF” and “VEGF-A” are used interchangeably and refer to the 165-amino acid vascular endothelial cell growth factor and related 121-, 189-, and 206-amino acid vascular endothelial cell growth factors, as described by Leung et al. Science, 246:1306 (1989), and Houck et al. Mol. Endocrin.; 5:1806 (1991), together with the naturally occurring allelic and processed forms thereof. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

The term “anti-VEGF antibody” refers to an antibody that binds to VEGF with sufficient affinity and specificity. Preferably, the anti-VEGF antibody of the invention can be used as a therapeutic agent in targeting and interfering with diseases or conditions wherein the VEGF activity is involved. An anti-VEGF antibody will usually not bind to other VEGF homologues such as VEGF-B or VEGF-C, nor other growth factors such as PIGF, PDGF or bFGF. A preferred anti-VEGF antibody is a monoclonal antibody that binds to the same epitope as the monoclonal anti-VEGF antibody A4.6.1 produced by hybridoma ATCC HB 10709. More preferably the anti-VEGF antibody is a recombinant humanized anti-VEGF monoclonal antibody generated according to Presta et al. (1997) Cancer Res. 57:4593-4599, including but not limited to the antibody known as bevacizumab (BV; Avastin®).

The anti-VEGF antibody “Bevacizumab (BV)”, also known as “rhuMAb VEGF” or “Avastin®”, is a recombinant humanized anti-VEGF monoclonal antibody generated according to Presta et al. (1997) Cancer Res. 57:4593-4599. It comprises mutated human IgG1 framework regions and antigen-binding complementarity-determining regions from the murine anti-hVEGF monoclonal antibody A4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated.

The term “VEGF antagonist” refers to a molecule capable of neutralizing, blocking, inhibiting, abrogating, reducing or interfering with VEGF activities including its expression and its binding to one or more VEGF receptors. VEGF antagonists include anti-VEGF antibodies and antigen-binding fragments thereof, receptor molecules and derivatives which bind specifically to VEGF thereby sequestering its binding to one or more receptors, anti-VEGF receptor antibodies and VEGF receptor antagonists such as small molecule inhibitors of the VEGFR tyrosine kinases. VEGF antagonists also include compositions that inhibit the expression or secretion of VEGF in or by a cell.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “transformation” and “transfection” mean the introduction of a nucleic acid, e.g., an expression vector, into a recipient cell including introduction of a nucleic acid to the chromosomal DNA of said cell.

“Mammal” for purposes of treatment refers to any animal classified as a mammal, including humans, domestic and farm animals, and zoo, sports, or pet animals, such as dogs, horses, cats, cows, etc. Preferably, the mammal is human.

The expression “therapeutically effective amount” refers to an amount of the antagonist which is effective for preventing, ameliorating or treating the autoimmune disease in question.

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

In addition, as used herein, the term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, the term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δBZ-L-ornithine, N-δ-Boc-N-α-CBZ-L-ornithine, Boc-p-nitro-L-phenylalanine, Boc-hydroxyproline, and Boc-L-thioproline.

The word “or” as used herein means any one member of a particular list and also includes any combination of members of that list.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

As used herein, “reverse analog” or “reverse sequence” refers to a peptide having the reverse amino acid sequence as another, reference, peptide. For example, if one peptide has the amino acid sequence ABCDE, its reverse analog or a peptide having its reverse sequence is as follows: EDCBA.

By “sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

By “modulate” is meant to alter, by increase or decrease.

By “normal subject” is meant an individual who does not have end-stage liver disease or does not have end-stage liver disease complications.

By an “effective amount” of a compound as provided herein is meant a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

By “treat” is meant to administer a compound or molecule of the invention to a subject, such as a human or other mammal (for example, an animal model), that has end-stage liver disease or end-stage liver disease complications, in order to prevent or delay a worsening of the effects of the disease or condition, or to partially or fully reverse the effects of the disease.

“Treatment” refers to both therapeutic treatment and prophylactic or preventative measures. Those in need of treatment include those already with the disease or disorder as well as those in which the disease or disorder is to be prevented. Hence, the mammal may have been diagnosed as having the disease or disorder or may be predisposed or susceptible to the disease.

By “prevent” is meant to minimize the chance that a subject develops end-stage liver disease or end-stage liver disease complications.

By “specifically binds” is meant that an antibody recognizes and physically interacts with its cognate antigen (for example, a c-Met polypeptide) and does not significantly recognize and interact with other antigens; such an antibody may be a polyclonal antibody or a monoclonal antibody, which are generated by techniques that are well known in the art.

By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for angiogenic nucleic acids (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the angiogenic nucleic acid to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

By “specifically hybridizes” is meant that a probe, primer, or oligonucleotide recognizes and physically interacts (that is, base-pairs) with a substantially complementary nucleic acid (for example, a c-met nucleic acid) under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-C1, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

An antagonist “which binds” an antigen of interest, e.g. VEGF, is one capable of binding that antigen with sufficient affinity and/or avidity such that the antagonist is useful as a therapeutic agent for targeting the antigen or a cell expressing the antigen:

An “isolated” antagonist is one which has been identified and separated and/or recovered from a component of its natural environment. Contaminant components of its natural environment are materials which would interfere with diagnostic or therapeutic uses for the antagonist, and may include enzymes, hormones, and other proteinaceous or nonproteinaceous solutes. In preferred embodiments, the antagonist will be purified (1) to greater than 95% by weight of antagonist as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antagonist includes the antagonist in situ within recombinant cells since at least one component of the antagonist's natural environment will not be present. Ordinarily, however, isolated antagonist will be prepared by at least one purification step.

The term “prodrug” as used in this application refers to a precursor or derivative form of a pharmaceutically active substance that is less cytotoxic to liver cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

A “liposome” is a small vesicle composed of various types of lipids, phospholipids and/or surfactant which is useful for delivery of a drug (such as the antagonists disclosed herein and, optionally, a chemotherapeutic agent) to a mammal. The components of the liposome are commonly arranged in a bilayer formation, similar to the lipid arrangement of biological membranes.

The term “intravenous infusion” refers to introduction of a drug into the vein of an animal or human patient over a period of time greater than approximately 5 minutes, preferably between approximately 30 to 90 minutes, although, according to the invention, intravenous infusion is alternatively administered for 10 hours or less.

The term “intravenous bolus” or “intravenous push” refers to drug administration into a vein of an animal or human such that the body receives the drug in approximately 15 minutes or less, preferably 5 minutes or less.

The term “subcutaneous administration” refers to introduction of a drug under the skin of an animal or human patient, preferable within a pocket between the skin and underlying tissue, by relatively slow, sustained delivery from a drug receptacle. The pocket may be created by pinching or drawing the skin up and away from underlying tissue.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is preferably less than approximately 15 minutes, more preferably less than 5 minutes, and most preferably less than 60 seconds. Administration is preferably within a pocket between the skin and underlying tissue, where the pocket is created, for example, by pinching or drawing the skin up and away from underlying tissue.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### J. REFERENCES

Adamis, A. P., D. T. Shima, et al. (1996) “Inhibition of vascular endothelial growth factor prevents retinal ischemia-associated iris neovascularization in a nonhuman primate.” Arch Opthalmol 114(1): 66-71.
Aiello, L. P., R. L. Avery, et al. (1994). “Vascular endothelial growth factor in ocular fluid of patients with diabetic retinopathy and other retinal disorders.” N Engl J Med 331(22): 1480-7.
Berkman, R. A., M. J. Merrill, et al. (1993). “Expression of the vascular permeability factor/vascular endothelial growth factor gene in central nervous system neoplasms.” J Clin Invest 91(1): 153-9.
Brown, L. F., B. Berse, et al. (1995). “Expression of vascular permeability factor (vascular endothelial growth factor) and its receptors in breast cancer.” Hum Pathol 26(1): 86-91.
Brown, L. F., B. Berse, et al. (1993). “Expression of vascular permeability factor (vascular endothelial growth factor) and its receptors in adenocarcinomas of the gastrointestinal tract.” Cancer Res 53(19): 4727-35.
Carmeliet, P., V. Ferreira, et al. (1996). “Abnormal blood vessel development and lethality in embryos lacking a single VEGF allele.” Nature 380(6573): 435-9.
Carmeliet, P. and R. K. Jain (2000). “Angiogenesis in cancer and other diseases.” Nature 407(6801): 249-57.
Chen, Y., C. Wiesmann, et al. (1999). “Selection and analysis of an optimized anti-VEGF antibody: crystal structure of an affinity-matured Fab in complex with antigen.” J Mol Biol 293(4): 865-81.
Chien, S., S. Li, et al. (1998). “Effects of mechanical forces on signal transduction and gene expression in endothelial cells.” Hypertension 31(1 Pt 2): 162-9.
Cicha, I., M. Goppelt-Struebe, et al. (2007). “Pharmacological inhibition of RhoA signaling prevents connective tissue growth factor induction in endothelial cells exposed to non-uniform shear stress.” Atherosclerosis.
Civelekoglu, G., Y. Tardy, et al. (1998). “Modeling actin filament reorganization in endothelial cells subjected to cyclic stretch.” Bull Math Biol 60(6): 1017-37.
Cooley, B. C., C. Y. Chen, et al. (2007). “Increased venous versus arterial thrombosis in the Factor V Leiden mouse.” Thromb Res 119(6): 747-51.
Dvorak, H. F., L. F. Brown, et al. (1995). “Vascular permeability factor/vascular endothelial growth factor, microvascular hyperpermeability, and angiogenesis.” Am J Pathol 146(5): 1029-39.
Faries, P. L., D. I. Rohan, et al. (2002). “Vascular smooth muscle cells derived from atherosclerotic human arteries exhibit greater adhesion, migration, and proliferation than venous cells.” J Surg Res 104(1): 22-8.
Ferrara, N. (1999). “Molecular and biological properties of vascular endothelial growth factor.” J Mol Med 77(7): 527-43.
Ferrara, N., K. Carver-Moore, et al. (1996). “Heterozygous embryonic lethality induced by targeted inactivation of the VEGF gene.” Nature 380(6573): 439-42.
Ferrara, N., H. Chen, et al. (1998). “Vascular endothelial growth factor is essential for corpus luteum angiogenesis.” Nat Med 4(3): 336-40.
Ferrara, N. and T. Davis-Smyth (1997). “The biology of vascular endothelial growth factor.” Endocr Rev 18(1): 4-25.
Ferrara, N., K. Houck, et al. (1992). “Molecular and biological properties of the vascular endothelial growth factor family of proteins.” Endocr Rev 13(1): 18-32.
Folkman, J. and M. Klagsbrun (1987). “Angiogenic factors.” Science 235(4787): 442-7.
Frangos, S. G., R. Knox, et al. (2001). “The integrin-mediated cyclic strain-induced signaling pathway in vascular endothelial cells.” Endothelium 8(1): 1-10.
Fung, Y. C. and S. Q. Liu (1993). “Elementary mechanics of the endothelium of blood vessels.” J Biomech Eng 115(1): 1-12.
Ganguli, A., L. Persson, et al. (2005). “Distinct NF-kappaB regulation by shear stress through Ras-dependent IkappaBalpha oscillations: real-time analysis of flow-mediated activation in live cells.” Circ Res 96(6): 626-34.
Garcia-Cardena, G., J. Comander, et al. (2001). “Biomechanical activation of vascular endothelium as a determinant of its functional phenotype.” Proc Natl Acad Sci USA 98(8): 4478-85.
Gerber, H. P., T. H. Vu, et al. (1999). “VEGF couples hypertrophic cartilage remodeling, ossification and angiogenesis during endochondral bone formation.” Nat Med 5(6): 623-8.
Guerrin, M., H. Moukadiri, et al. (1995). “Vasculotropin/vascular endothelial growth factor is an autocrine growth factor for human retinal pigment epithelial cells cultured in vitro.” J Cell Physiol 164(2): 385-94.
Hirakawa, M., M. Oike, et al. (2004). “Sequential activation of RhoA and FAK/paxillin leads to ATP release and actin reorganization in human endothelium.” J Physiol 558(Pt 2): 479-88.
Iba, T., S. Maitz, et al. (1991). “Effect of cyclic stretch on endothelial cells from different vascular beds.” Circ Shock 35(4): 193-8.
Jones, P. T., P. H. Dear, et al. (1986). “Replacing the complementarity-determining regions in a human antibody with those from a mouse.” Nature 321(6069): 522-5.
Kim, K. J., B. Li, et al. (1993) “Inhibition of vascular endothelial growth factor-induced angiogenesis suppresses tumour growth in vivo.” Nature 362(6423): 841-4.
Kisucka, J., C. E. Butterfield, et al. (2006). “Platelets and platelet adhesion support angiogenesis while preventing excessive hemorrhage.” Proc Natl Acad Sci USA 103(4): 855-60.
Kuebler, W. M., U. Uhlig, et al. (2003). “Stretch activates nitric oxide production in pulmonary vascular endothelial cells in situ.” Am J Respir Crit. Care Med 168(11): 1391-8.
Lehoux, S., Y. Castier, et al. (2006). “Molecular mechanisms of the vascular responses to haemodynamic forces.” J Intern Med 259(4): 381-92.
Li, Y., J. Zheng, et al. (2004). “Mechanisms of shear stress-induced endothelial nitric-oxide synthase phosphorylation and expression in ovine fetoplacental artery endothelial cells.” Biol Reprod 70(3): 785-96.
Li, Y., J. Zheng, et al. (2005). “Effects of pulsatile shear stress on signaling mechanisms controlling nitric oxide production, endothelial nitric oxide synthase phosphorylation, and expression in ovine fetoplacental artery endothelial cells.” Endothelium 12(1-2): 21-39.
Li, Y. S., J. H. Haga, et al. (2005). “Molecular basis of the effects of shear stress on vascular endothelial cells.” J Biomech 38(10): 1949-71.
Liang, F., N. Huang, et al. (2002). “Shear stress induces interleukin-8 mRNA expression and transcriptional activation in human vascular endothelial cells.” Chin Med J (Engl) 115(12): 1838-42.
Lopez, P. F., B. D. Sippy, et al. (1996). “Transdifferentiated retinal pigment epithelial cells are immunoreactive for vascular endothelial growth factor in surgically excised age-related macular degeneration-related choroidal neovascular membranes.” Invest Opthalmol Vis Sci 37(5): 855-68.
Malek, A. M., S. Izumo, et al. (1999). “Modulation by pathophysiological stimuli of the shear stress-induced up-regulation of endothelial nitric oxide synthase expression in endothelial cells.” Neurosurgery 45(2): 334-44; discussion 344-5.
Mattern, J., R. Koomagi, et al. (1996). “Association of vascular endothelial growth factor expression with intratumoral microvessel density and tumour cell proliferation in human epidermoid lung carcinoma.” Br J Cancer 73(7): 931-4.
Mazzag, B. M., J. S. Tamaresis, et al. (2003). “A model for shear stress sensing and transmission in vascular endothelial cells.” Biophys J 84(6): 4087-101.
Melnyk, O., M. A. Shuman, et al. (1996). “Vascular endothelial growth factor promotes tumor dissemination by a mechanism distinct from its effect on primary tumor growth.” Cancer Res 56(4): 921-4.
Morawietz, H., R. Talanow, et al. (2000). “Regulation of the endothelin system by shear stress in human endothelial cells.” J Physiol 525 Pt 3: 761-70.
Nagel, T., N. Resnick, et al. (1999). “Vascular endothelial cells respond to spatial gradients in fluid shear stress by enhanced activation of transcription factors.” Arterioscler Thromb Vasc Biol 19(8): 1825-34.
Noris, M., M. Morigi, et al. (1995). “Nitric oxide synthesis by cultured endothelial cells is modulated by flow conditions.” Circ Res 76(4): 536-43.
Oberg-Welsh, C., S. Sandler, et al. (1997). “Effects of vascular endothelial growth factor on pancreatic duct cell replication and the insulin production of fetal islet-like cell clusters in vitro.” Mol Cell Endocrinol 126(2): 125-32.
Presta, L. G., H. Chen, et al. (1997). “Humanization of an anti-vascular endothelial growth factor monoclonal antibody for the therapy of solid tumors and other disorders.” Cancer Res 57(20): 4593-9.
Quist, W. C., C. C. Haudenschild, et al. (1992). “Qualitative microscopy of implanted vein grafts. Effects of graft integrity on morphologic fate.” J Thorac Cardiovasc Surg 103(4): 671-7.
Quist, W. C. and F. W. LoGerfo (1992). “Prevention of smooth muscle cell phenotypic modulation in vein grafts: a histomorphometric study.” J Vasc Surg 16(2): 225-31.
Ranjan, V., Z. Xiao, et al. (1995). “Constitutive NOS expression in cultured endothelial cells is elevated by fluid shear stress.” Am J Physiol 269(2 Pt 2): H550-5.
Riechmann, L., M. Clark, et al. (1988). “Reshaping human antibodies for therapy.” Nature 332(6162): 323-7.
Seebach, J., G. Donnert, et al. (2007). “Regulation of endothelial barrier function during flow-induced conversion to an arterial phenotype.” Cardiovasc Res 75(3): 596-607.
Shiu, Y. T., S. Li, et al. (2003). “Shear stress-induced c-fos activation is mediated by Rho in a calcium-dependent manner.” Biochem Biophys Res Commun 303(2): 548-55.
Shyy, J. Y. and S. Chien (2002). “Role of integrins in endothelial mechanosensing of shear stress.” Circ Res 91(9): 769-75.
Siemeister, G., G. Martiny-Baron, et al. (1998). “The pivotal role of VEGF in tumor angiogenesis: molecular facts and therapeutic opportunities.” Cancer Metastasis Rev 17(2): 241-8.
Sondell, M., G. Lundborg, et al. (1999). “Vascular endothelial growth factor has neurotrophic activity and stimulates axonal outgrowth, enhancing cell survival and Schwann cell proliferation in the peripheral nervous system.” J Neurosci 19(14): 5731-40.
Thamilselvan, V. and M. D. Basson (2005). “The role of the cytoskeleton in differentially regulating pressure-mediated effects on malignant colonocyte focal adhesion signaling and cell adhesion.” Carcinogenesis 26(10): 1687-97.
Thoumine, O., R. M. Nerem, et al. (1995). “Oscillatory shear stress and hydrostatic pressure modulate cell-matrix attachment proteins in cultured endothelial cells.” In Vitro Cell Dev Biol Anim 31(1): 45-54.
Upchurch, G. R., Jr., A. J. Banes, et al. (1989). “Differences in secretion of prostacyclin by venous and arterial endothelial cells grown in vitro in a static versus a mechanically active environment.” J Vasc Surg 10(3): 292-8.
Verhoeyen, M., C. Milstein, et al. (1988). “Reshaping human antibodies: grafting an antilysozyme activity.” Science 239(4847): 1534-6.
Wagner, W. H., R. M. Henderson, et al. (1988). “Differences in morphology, growth rate, and protein synthesis between cultured arterial and venous endothelial cells.” J Vasc Surg 8(4): 509-19.
Warren, R. S., H. Yuan, et al. (1995). “Regulation by vascular endothelial growth factor of human colon cancer tumorigenesis in a mouse model of experimental liver metastasis.” J Clin Invest 95(4): 1789-97.
Wittstein, I. S., W. Qiu, et al. (2000). “Opposite effects of pressurized steady versus pulsatile perfusion on vascular endothelial cell cytosolic pH: role of tyrosine kinase and mitogen-activated protein kinase signaling.” Circ Res 86(12): 1230-6.
Wong, A. P., N. Nili, et al. (2005). “In vitro differences between venous and arterial-derived smooth muscle cells: potential modulatory role of decorin.” Cardiovasc Res 65(3): 702-10.
Yano, Y., J. Geibel, et al. (1997). “Cyclic strain induces reorganization of integrin alpha 5 beta 1 and alpha 2 beta 1 in human umbilical vein endothelial cells.” J Cell Biochem 64(3): 505-13.

### K. EXAMPLES

The following examples are put forth so as to provide those of ordinary skill in the art with a complete disclosure and description of how the compounds, compositions, articles, devices, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### 1. Example 1

Two groups of rats can be used to demonstrate the effects of bevacizumab on the formation of portal-systemic collaterals in a rat model of cirrhosis. Rats can first be injected with Matrigel™ collagen matrix (BD Biosciences, San Jose, Calif.). Cirrhosis can then be induced in each of the two groups of rats by administering CCl₄ to the rats. An I.P. sham injection can be administered to the control rats and bevacizumab can be administered ip to the non-control rat group. Necropsy and quantification of variceal formation can then be assessed in each of the two groups.

### 2. Example 2

A patient with advanced liver disease who has demonstrated continued and ongoing progression of end stage liver disease or patients early in their disease course who have begun to develop end-organ complications as a consequence of increased venous capacitance can also be assessed.

Candidates for therapy according to this example include patients with a MELD score>15. Other criteria can include patients with decompensated liver disease or evidence of heptorenal syndrome (either Type I or Type II,), patients being considered for primary prophylaxis against variceal bleeding, patients being considered for prevention of recurrent variceal hemorrhage, patients in whom a reduction in portal pressures is a desired outcome, and patients with refractory ascites and abnormalities in sodium handling. For these purposes, Type I HRS would be defined as rapid and progressive impairment of renal function, doubling of the initial serum creatinine to a level greater than 2.5 mg/dL or a 50% reduction of initial 24 h creatinine clearance to a level lower than 20 mL/min in less than 2 weeks. Diagnosis of HRS can be determined using the criteria proposed by the International Ascites Club: (i) low GFR, as indicated by serum creatinine>1.5 mg/dL or 24 h creatinine clearance<40 mL/min; (ii) absence of shock, ongoing bacterial infection, fluid losses and treatment with nephrotoxic drugs; (iii) no improvement of renal function following diuretic withdrawal and plasma volume expansion; (iv) proteinuria<500 mg/day; and (v) no ultrasonographic evidence of renal parenchymal disease or urinary tract obstruction. Type II HRS can be defined as impairment in renal function (serum creatinine>1.5 mg /dL) that does not meet criteria for Type I.

In patients with Type I HRS, monitoring can occur in the intensive care unit to assess the therapeutic effects and potential side-effects of the treatment. The vital signs can be assessed every 4 h, and central venous pressure and urine output can be measured every 8 h. Blood samples can be taken before the start of therapy and every 2 days throughout the treatment, for tests of liver and renal functions. To rule out prerenal failure, a central venous catheter can be inserted. Patients can then receive i.v. albumin infusion, 20 g/day and fresh frozen plasma (FFP) 150 mL every 8 h, until central venous pressure reached the upper normal range (10-12 cm of H₂O). The patients can receive midodrine 10 mg tid or low dose levophed (3-5 mcg/min) to maintain renal perfusion pressure. Throughout the study, sodium and fluid intake can be restricted and maintained at 40 mEq/day and 1 L/day, respectively. For tense ascites, 3-5 L paracentesis can be performed, along with infusion of 8 g of albumin for each liter of ascitic fluid removed. The mean arterial pressure can be calculated as diastolic pressure plus one-third of pulse pressure. Serum and urinary creatinine can be determined by a rate blanked and compensated Jaffe reaction. Creatinine clearance (mL/min) can be calculated as a product of urinary creatinine (mg/dL) and 24-h urine volume divided by serum creatinine (mg/dL) multiplied by 1440.

The results can be expressed as mean±standard error of the mean. The analysis of the results can be performed by using a paired Student's t-test and a non-parametrical Mann-Whitney U-test using the SPSS 9.01 statistical package. A value of P<0.05 can be taken as significant. The results can be analyzed at baseline, day 4, day 8 and finally at day 15 of the study. Survival and therapy failures can be analyzed by the Kaplan-Meier method and can be compared to patients receiving standard therapy alone using the Breslow and log-rank tests.

In patients with gastrointestinal varices, therapy can be initiated in those with (1) end-stage liver disease confirmed by biopsy; (2) endoscopic documentation of variceal hemorrhage (actively bleeding varix or non-bleeding varices without other lesions) requiring at least one unit of blood transfusion; (3) arrest of acute variceal hemorrhage either spontaneously or by use of intravenous vasopressin and/or somatostatin and/or balloon tamponade and/or homeostatic sessions of endosclerotherapy, banding, or ligation; (4) patency of the splanchnic venous system and hepatopetal portal flow (according to Nordlinger's classification); (5) eligible for either surgical shunt, liver transplant, transjugular intrahepatic portosystemic shunt (TIPS), or endosclerotherapy/banding/ligation (6) absence of non-hepatic malignanceis); (7) willing to return for regular follow-up.

Patients can be administered therapy once they are fully resuscitated, hemodynamically stable, and have demonstrated evidence of mucosal healing. Variceal rebleeding within 2 years of first treatment can be considered as the primary measure of patient outcome. Patients can continue to receive standard therapy as warranted.

A complete medical history can be obtained for each patient, and particular notice can be taken of previous episodes of gastrointestinal bleeding and evidence of either primary or post hemorrhagic hepatic decomposition (jaundice, ascites or edema). Routine laboratory tests can be performed to evaluate liver function. Overall assessment of the severity of liver disease can be graded according to the MELD system. Serum alpha-fetoprotein assessment and computed tomography of the abdomen can be routinely performed in order to screen for the presence of hepatocellular carcinoma. The presence of esophageal varices can be assessed through endoscopic examination. Criteria used for classifying the endoscopic findings can be based on the General Rules for Recording Endoscopic Findings on Esophageal Varices compiled by the Japanese Research Society for Portal Hypertension

Cerebral function can be assessed through a complete neurological examination, taking into account mental state, asterixis, electroencephalographic findings (EEG), the trail making test, or the “Cancelling A′ s” test Encephalopathy can be considered “acute” if it was precipitated by gastrointestinal bleeding, heavy drinking, pharmacological or dietary imbalances, of brief duration and easily controlled with elimination of the precipitating cause. Encephalopathy can be deemed “chronic” if it was spontaneous, of long duration and more difficult to manage.

In the evaluation of hospital mortality and early complications, the first 30 d after the initial treatment can be defined as the post-treatment period. In the post-treatment period, esophageal endoscopy can be performed on each patient. Patients can be initiated on therapy if they are free of complications, such as mucosal ulcerations, symptomatic stricture, severe esophagitis, fever, and pneumonia. In the presence of complications, an upper endoscopy can be performed at intervals of seven to ten days and therapy initiated only when complications were resolved.

During the follow-up period, patients can be checked at 1, 3 and 6 more after the first endoscopy and then at least twice yearly, on an outpatient, unless recurrent hemorrhage occurred. At each visit, liver function can be evaluated following a complete medical examination and laboratory tests. Assessment of the neurological status can be performed using the above-mentioned criteria. An EEG can be performed at least once a year as indicated. If the etiology of the portal hypertension stems from alcohol use, a return to drinking can be ascertained based on patients' statements, our own assessment and information from relatives. Continued drinking can be defined as daily consumption in excess of 1 liter wine and/or spirits. All patients would be on a 10-meq sodium and protein-balanced diet (1 g protein/kg body wt) and undergoing lactulose prophylactic treatment: the initial dose would be 60 g/d in 3 separate doses and adjusted thereafter to induce at least 1 bowel movement per day.

Eradication can be defined as the absence of varices or the presence of F1 white varices. Rebleeding can be defined as hemorrhage due to esophago-gastric varices and/or congestive gastropathy, requiring at least 1 unit blood transfusion and was designated as being from varices if this was supported by endoscopic findings. The treatment of choice for variceal rebleeding can be emergency endoscopic variceal band ligation. Chronic rebleeding from congestive gastropathy can be treated with beta-blocking therapy. Rebleeding due to peptic ulcer can be recorded separately.

Initial and subsequent data for the patients can be collected on a dedicated spreadsheet (Excel, Microsoft Corp., Delaware, USA) for personal computer input (Macintosh G4, Apple Computer Inc.) and subsequent analysis (Statistica-Mac, Statsoft, Tulsa Okla., USA). Survival and therapy failures can be analyzed by the Kaplan-Meier method and can be compared to patients receiving standard therapy alone using the Breslow and log-rank tests. Comparison between groups can be made by Chi-square test for proportions and Student's t-test for the means.

For all studies, there can be no exclusion based on age, sex, etiology, or duration of portal hypertension. Major exclusion criteria can be based on concerns of general safety such as a history of recent surgery or active peptic ulcer disease.

Additional patients can be those biopsy-proven patients with end-stage liver disease of any etiology who were well compensated, without a history of ascites, or diuretic use. These patients can be termed pre-ascitic patients and the purpose of additional investigations can be to establish the impact of anti-angiogenic therapy as prophylaxis against the development of portal systemic collaterals, circulatory derangements, and effects on sodium balance that invariably occur as a natural progression of end stage liver disease. All patients can be stable and would have abstained from alcohol for at least six months prior to entry. Patients with intrinsic renal or cardiovascular disease on history or physical exam with abnormal urinalyisis, chest x-ray, or electrocardiograph can be excluded from further study. These patients can be compared with an appropriate age and diseased-matched control populations. Patients can be placed on a 200 mmol sodium, 1.5 L fluid per day diet. Twenty-four hour urine collections can be made at the end of each week as an estimate of sodium handling. Daily weights can be recorded throughout the study period.

Once a baseline renal sodium retaining state had been obtained, preascitic patients can have baseline hemodynamic and renal studies performed. In the evening, at 10:00 pm prior to the baseline studies, all study subjects can receive lithium carbonate 300 mg orally for the measurement of lithium clearance as a measure of proximal tubular reabsorption of sodium. Assessments of baseline renal function, systemic hemodynammics, and neurohormonal factors can be performed at baseline. Shortly after 8 00 am the day following baseline measurements, an intravenous catheter can be inserted. After two hours of bedrest in the supine position, blood can be collected from all study subjects via an indwelling catheter without a tourniquet for measurement of serum electrolytes, plasma rennin activity (PRA), plasma angiotensin II, and aldosterone levels. Measurements of inulin clearance, a measurement of glomerular filtration rate (GFR), p-aminohippurate clearance, and index of renal plasma flow (RPF), lithium clearance, and urinary sodium excretion can be made for two periods of one hour each, with patients voiding while supine. Mean arterial pressure (MAP) and heart rates can be assessed hourly during the renal studies. In the afternoon at 2:00 pm, after fasting for at least six hours, study subjects can be transferred to the Nuclear Cardiology Department for measurement of central blood volume and systemic hemodynamics using radionuclide angiography. An additional 24 hour urine collection can be made for urinary sodium excretion the same day.

After a period of three months, while maintained on the 200 mmol sodium, 1.5 liter fluid per day, diet, the hemodynamic and renal studies can be repeated after anti-angiogenic therapy. Study subjects can receive lithium carbonate, 300 mg, at 10 00 pm on the evening prior to the repeat studies. Subsequent measurements of renal sand systemic hemodynamics, sodium excretion (both during the two hour can be repeated in exactly the same way as baseline measurements.

Inulin and p-aminohippurate clearances can be corrected for body surface are and expressed per 1.73 m2. Renal vascular resistance (RVR) for each clearance period=MAP+renal blood flow; renal blood flow=RPF/(1-packed cell volume). Proximal tubular reabsorption of sodium can be calculated using lithium clearance and GFR, whereas the distal tubular reabsorption of sedum can be calculated from inulin clearance, serum sodium concentrations, urinary sodium concentrations, and urinary volume.

The end diastolic, end systolic, and central blood volumes can be measured directly by radionuclide angiography. Stroke volume, cardiac output, and systemic vascular resistance (SVR) can be calculated from standard formulae. All volume measuremtns can be corrected for body surface area using a subject's height and weight. Likewise, cardiac output can be corrected for body surface area to yield cardiac index.

All results can be expressed as mean (SEM). For independent variables, paired and unpaired Student's t tests can be used to analyze two means of each variable. Differences can be considered significant if the null hypothesis could be rejected at the 0.05 probability level.

To study the effects of anti-angiogenic therapy on refractory ascites, end-stage liver disease patients with tense ascites and spontaneous bacterial peritonitis (SBP) who are admitted to the hospital can be prospectively enrolled in this study. The diagnosis of end-stage liver disease can be based on clinical, laboratory, and ultrasonographic findings. The diagnosis of SBP can be made when the white blood cell count in tapped ascitic fluid was over 500/mm³ and polymorphonuclear cell count was over 50% (>250/mm³) and secondary bacterial peritonitis, tuberculous peritonitis, peritonitis due to pancreatitis, and secondary ascites due to carcinomatosis can be excluded. A positive ascitic fluid culture would not be considered necessary for the diagnosis, because culture-negative neutrocytic ascites is accepted as a variant of SBP. At enrollment, patients with infection in other sites, septic shock, heart failure, grade 3 or 4 of hepatic encephalopathy, gastrointestinal bleeding, chronic renal failure, or serum creatinine level of more than 3.0 mg/dL, and any disease (e.g. advanced malignancy) that could affect the short-term prognosis would be excluded.

After baseline measurements, patients can be alternately assigned to each group with the first patient assigned to Group 1 and the next patient to Group 2, and so forth. In Group 1, LVP can be carried out within 24 h after the diagnosis of SBP. After large volume paracentesis (LVP) can be carried out, diuretics (spironolactone alone or in combination with furosemide) can be administered, if necessary, to prevent reaccumulation of ascites. LVP would be defined as a drainage of ascitic fluid of more than 4 liters in a single tap or loss of shifting dullness after paracentesis. To expand plasma volume, 6-8 g of albumin (25% human albumin solution) per 1 liter of removed ascitic fluid can be administered intravenously. In Group 2, oral diuretics (spironolactone alone or in combination with furosemide) can be administered to all patients, and albumin would be intravenously administered to patients whose serum albumin levels were less than 3.0 g/dL in conjunction with anti-angiogenic therapy. The starting dose of spironolactone would be 100 or 200 mg/day and furosemide can be started at a dose of 40 or 80 mg/day. These doses can be increased, in a stepwise fashion until the highest recommended doses was achieved (400 mg/day of spironolactone, and 160 mg/day of furosemide) if there was no response.

In both groups, 2 g of cefotaxime can be administered twice daily. If serum creatinine concentration was higher than 2.0 mg/dL, 1 g of cefotaxime can be administered twice daily. In both groups, patients will not receive secondary prophylaxis with oral antibiotics after discharge from the hospital.

Daily physical examination will be carried out to observe the changes of clinical symptoms and signs. A series of diagnostic paracentesis and blood tests can be done at 30 days after treatment. Resolution of SBP will be defined as a disappearance of symptoms and signs, and polymorphonuclear cell count in ascitic fluid of less than or equal to 250/mm³. Patients can also be compared with regard to MELD score, MELD score acceleration, and serum creatinine 1n patients who did not respond to cefotaxime, antibiotic treatment can be modified according to the in vitro susceptibility of the isolated organism or was modified empirically in patients with negative blood and ascitic-fluid cultures.

To appraise treatment-related complications, the definitions will be as follows: newly developed hepatic encephalopathy and aggravation of preexisting hepatic encephalopathy would be defined as complications of hepatic encephalopathy. Renal impairment will be defined as elevation of serum creatinine concentration to higher than 1.5 mg/dL in patients without preexisting renal insufficiency, and elevation of more than 50% of the baseline level in patients with initial serum creatinine concentration of higher than 1.5 mg/dL. Hyponatremia will be defined as a decline of serum sodium concentration to lower than 130 mEq/L and a reduction of more than 5 mEq/L compared to the baseline level in patients with initial serum sodium concentration of higher than or equal to 130 mEq/L, and a decline of serum sodium concentration of more than 5 mEq/L in patients with initial serum sodium concentration of less than 130 mEq/L.

SPSS program (version 11.0) can be used for statistical analysis and a P-value 0.05 can be required for statistical significance, and all tests would be two-sided. Unless otherwise stated, results can be given as means±SEM (standard error mean) or frequencies. Continuous data such as age, blood tests, ascitic fluid analyzes, in-hospital days, symptom durations, and durations of antibiotic therapy can be compared between the two groups with Student's t-test or Mann-Whitney U-test, and categorical data such as gender ratio, symptom frequencies, resolution rates of SBP, and complication rates can be compared with Pearson x2 test or Fisher's exact test. Survival analysis can be performed by Kaplan-Meier method, and differences between the two groups can be assessed with the log-rank test. Surviving patients can be censored at the last clinical visit or interview.

To examine the effect of anti-angiogenic therapy on the reduction of portal pressures, individuals with end-stage liver disease (MELD>15) and esophageal varices can be selected consecutively for study. Only those patients would be taken who had either never bled, or who had experienced only one episode of variceal bleeding at least 4 wk before inclusion but did not receive any specific therapy for variceal bleeding in the form of endoscopic procedures or long term pharmacotherapy for portal hypertension before referral. Only those patients with a history of recent bleed would be included who, on endoscopy, did not have any sign of active bleeding. Criteria adopted for exclusion could be bronchial asthma, significant cardiac diseases, hypertension, renal disease (serum creatinine>1.5 mg/dl or suggestive ultrasonographic (USG) change), and age<15 yr or >65 yr. Additional criteria can be treatment by endoscopic sclerotherapy, variceal ligation, any surgery for portal hypertension, β or α1-adrenergic blockers, diuretics, and nitrates. Disabling ascites could be treated with paracentesis to avoid the confounding effect of diuretics (including spironolactone) on portal pressure and to avoid hypotension and renal compromise. Patients would be hospitalized for the duration of the study. All patients would have abstained from alcohol for 3 months before as well as during the study. All patients with ascites would be put on a low sodium (50 mEq/L) diet. The patients without ascites would be considered as nonascitic end stage liver disease patients if they had no history of abdominal swelling and if previous (whenever available) as well as present ultrasonography did not reveal ascites. The patients would be investigated by liver function tests including enzyme studies, viral markers, upper GI endoscopy, ultrasonography with Doppler, prothrombin time, and liver biopsy to confirm the diagnosis of end stage liver disease. Compatible ultrasonographic finding (contracted liver with ascites) in the presence of esophageal varices and HVPG≧12 mm Hg would be taken as evidence of end stage liver disease in cases in which liver biopsy could not be done. Variceal grading was adopted as per the Japanese Research Society.

After an overnight fast, the patients would be taken to the catheterization laboratory in the morning, where hemodynamic investigations would be carried out using standard procedures. Tracings of the pressure measurement would be obtained and independently corroborated by an observer who was not involved in the hemodynamic measurement. Measurements would be made in triplicate and the mean taken in each case. HVPG, which is equivalent to portal venous pressure, is obtained as the difference between WHVP and FHVP.

The cases included would be randomized using computer-generated randomization sequence into two groups of patients, one on standard therapy with propranolol and the second receiving propranolol and anti-angiogenic therapy. After a baseline hemodynamic study, the patients either received anti-angioigenic therapy losartan) or propranolol (Inderal; ICI Pharmaceuticals India, Chemai, India) 40 mg b.i.d. (8 AM and 8 PM) p.o. The dose of propranolol would be further titrated by a physician supervising drug compliance to achieve a pulse rate reduction of 25% of baseline but not less than 55 beats/min. After 3 months, the hemodynamic study would be repeated.

Arterial pressure would be monitored by the Korotkoff method with the patient seated, and mean arterial blood pressure calculated as follows: mean arterial blood pressure=⅓ (pulse pressure)+diastolic blood pressure. Heart rate would be monitored by automatic recording during the hemodynamic studies and clinically during the interim period. Serum bilirubin, alanine and aspartate aminotransferase (ALT, AST), serum urea, and creatinine would be measured before and on day 14 after starting the drugs.

After discharge from the hospital, the patients would be asked to attend the clinic at intervals of 2 wk initially and then of 4 wk. In follow-up, patients with disabling ascites would be prescribed diuretics starting with a low dose. A close monitoring of the patients would be done to detect GI bleeding, worsening of renal parameters (serum urea, creatinine), bradycardia (<55 beats/min), and hypotension (systolic blood pressure<90 mm Hg). The patients would be asked to maintain a diary and to bring the empty medicine containers during their follow-up visits to check for compliance.

Results would be expressed as mean±SD. Correlation and regression, x2 test, Fisher's exact test, and single-factor ANOVA were used as required. A value of p<0.05 would be considered to be statistically significant. The anti-VEGF antibody used for therapy could be bevacizumab (Avastin.RTM., commercially available from Genentech, Inc.) or a variant thereof having improved binding affinity, inhibitory efficacy or pharmacokinetic properties.

Patients would be treated with a therapeutically effective dose of the antibody, for instance, a single dose of 1-2.5 mg/kg i.v. every two weeks (1.0 mg/kg/wk) administered systemically, intraperitoneally, or delivered directly into the portal venous system via a catheter-directed approach. Patients can also receive concomitant sorafenib, 400 mg p.o., taken twice daily. Patients would optionally continue to receive any background diuretic (spironolactone up to 400 mg/day alone or in combination with lasix 160 mg/day) beta blocker therapy (propranolol at a starting dose of 40 mg b.i.d.), along with i.v. or non-absorbable oral antibiotics (norfloxacin, 400 mg daily), intravenous or oral vasoconstrictors (midodrine, 10 mg t.i.d), bile acid therapy (13-15 mg/kg/day in 2-4 divided doses with food), and human albumin infusions.

Instead of Avastin, patients could also be treated with 50 mg zinc as zinc gluconate, zinc acetate, zinc sulfate, or zinc chloride three times daily with meals (breakfast, lunch, dinner) and disulfuram 250 mg nightly with a bedtime snack.

### 3. Example 3

A patient with symptomatic early hemorrhoidal disease who has failed conservative medical management. Candidates for therapy according to this example are those who suffer from symptomatic 2nd degree hemorrhoids (according to Goligher's classification), and who had undergone six months unsuccessful medical treatment which included diet, fibre and topical agents. Written informed consent was obtained from all patients. The patients of the first group would undergo standard therapy with sclerotherapy and rubber band ligation (SCL/RBL), alone or in combination, and the patients of the 2^{nd} group would undergo injection of the anti-angiogenic agent directly into the varix. For bevacizumab, a dose 1/10 that of the systemic dose, or other concentration, could be used in a volume of approximately 2-6 mL. A surgeon who was very experienced in the field of coloproctology would perform all the procedures.

All the patients were asked about their medical history and were then examined clinically as well as with proctosigmoidoscopy. When there was an indication, either colonoscopy or barium enema would be performed to exclude other possible causes of bleeding. In the SCL/RBL group, simultaneous sclerotherapy of smaller non-prolapsing haemorrhoidal piles and rubber band ligation of the larger prolapsing piles would be performed. Up to 2 legations, according to the well-known technique of Barron, would be performed during any one session. Sclerotherapy would be done by submucosal injection of a 5% phenol solution in almond oil, as described by Blanchard. The amount of sclerosing solution to be used would be 2 ml per pile and 2-6 ml in total per session. In the bevacizumab group, the same technique would be applied. All patients would be informed about possible immediate or later complications and were given written instructions for stool softening. They would all be examined after 4 weeks and afterwards in case of persistent or recurrent symptoms. After 4 years, all patients would be contacted and examined and their symptoms recorded.

Differences among treatment groups would be tested using the X² test, followed by pairwise comparisons. The unadjusted analyses would be reported with a significance level of 0.05. Statistical analyses would be conducted in SPSS 10.0 (SPSS Inc, Chicago, Ill., USA).

### 4. Example 4

A patient, who, owing to the patient's history, clinical examination, and the results of digital photoplethysmography, Doppler examination, and duplex examination, are found to have with varicose veins (C2-4, EP, ASP, PR). Candidates for therapy according to this example would have superficial varicose veins of 3 to 6 mm in diameter but competent saphenofemoral and saphenopopliteal junctions. The calibers of the varicose veins would be calculated in horizontal position using duplex ultrasound. Exclusion criteria would be pregnancy, acute thrombosis/phlebitis, thrombophilia, and peripheral arterial occlusive disease (ankle-brachial index<0.9).

First duplex sonography would be performed to document the compressibility, caliber, and venous blood flow in the varicose veins. Moreover, the venoarterial flow index as a quantitative pattern for venous hemodynamics would be calculated using duplex. Then the double-blinded injection would be carried out. Adhesive external compression bandages (for 2-3 days) and compression stockings (compression class 2) for 1 week would be applied.

One week after treatment, duplex ultrasound would be performed to document vein occlusion, compressibility, caliber, and venous blood flows in the treated vein and to calculate venoarterial flow index. Obliteration would be defined if the treated veins showed no compressibility and if no venous blood flow could be provoked.

Four weeks after treatment, the same morphologic criteria (compressibility, caliber, venous blood flows in the treated vein) and venoarterial flow index would be investigated using duplex sonography.

Twelve weeks later the same criteria and venoarterial flow index would be measured by duplex.

After inclusion, the subjects would be randomized into two groups. The first group could be treated with injection using 2 or 3% bevacizumab without preservatives) adapted to the caliber of the varicose veins. Veins of 3 to 4 mm in diameter would be treated with 2% solution and in those of 5- to 6-mm, anti-angiogenic therapy in a concentration of 3% would be injected.

Patients in the second group would receive placebo (normal saline) injections. The procedure would be double-blinded: neither the patients nor the physician performing the procedure know whether bevacizumab or placebo was injected.

After the procedure, external adhesive compression bandages would be applied for 2 to 3 days (2 days in case 2% liquid was used and 3 days if a patient received 3% liquid). Moreover, the patients would be treated with knee-length compression stockings for 1 week after the therapy.

A color-duplex scanner (Apogee 800, Advanced Technology Laboratories, Solingen, Germany) with a 7.5-MHz L40 linear array would be used for duplex measurements in the common femoral vein. These would be taken proximal to the saphenofemoral junction. The common femoral artery would be examined proximal to the bifurcation. Duplex sonography would be performed under standardized conditions in relaxed horizontal supine position with slightly elevated upper part of the body. First the accurate diameter (d=2r) of the common femoral vein and common femoral artery would be measured in the cross-section. Care would be taken to ensure that the positioning of the transducer on the skin was performed without any pressure to avoid any influence on the diameter of the vessels. Both cross-sectional and longitudinal measurements of the diameter of the vessel would then be repeated several times within 30 s to take the respiratory rhythm into account. Mean blood flow velocity (Vm) would be calculated in the longitudinal section during a time course of more than 30 s. With regard to backward and forward flow, the area under the curve (integral) would be determined. Volume flow (VF; venous flow volume and arterial flow volume) would be calculated from the formula VF=Vm×πr2 by the software of the duplex scanner. The quotient of venous flow volume by arterial flow volume would calculate the venoarterial flow index.

To prove the efficacy of this therapy, photographs would be taken and questionnaires were administered before treatment and at 1, 4, and 12 weeks after treatment. Three vascular surgeons blinded to treatment and study center would evaluate pre- and posttreatment photographs to determine overall disappearance on a scale of 1-5: 1=worse than before treatment, 2=no change, 3=minor disappearance, 4=moderate disappearance, 5=complete disappearance. The distributions of the venoarterial flow index would be presented as means±SD. For pairwise comparisons the two-sided Student's t test would be used. The global significance level would be fixed at α=0.05.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. A method of treating or preventing acute or chronic liver inflammation in a subject, comprising administering to the subject an effective amount of a dithiocarbamate.

23. The method of claim 22, wherein the dithiocarbamate is disulfuram.

24. The method of claim 22, wherein the dithiocarbamate is diethyldithiocarbamate.

25. The method of claim 22, further comprising separately administering to the subject a therapeutically effective amount of zinc, or a pharmaceutically acceptable salt thereof.

26. The method of claim 25, wherein the pharmaceutically acceptable salt of zinc is zinc gluconate, zinc acetate, zinc sulfate, or zinc chloride.

27. The method of claim 22, wherein the subject is a mammal.

28. The method of claim 27, wherein the mammal is a human.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. Use of a dithiocarbamate in the preparation of a medicament for the treatment of acute or chronic liver inflammation in a subject

41. The use of claim 39, wherein the medicament further comprises zinc, or a pharmaceutically acceptable salt thereof.

42. The use of claim 41, wherein the dithiocarbamate is disulfuram.

43. The use of claim 41, wherein the dithiocarbamate is diethyldithiocarbamate.

44. The use of claim 41, wherein the pharmaceutically acceptable salt of zinc is zinc gluconate.

45. The use of claim 40, wherein the subject is a mammal.

46. The use of claim 45, wherein the mammal is a human.
